(19) United States
(12) Patent Application Publication
(10) Pub. No.: US 2016/0209416 A1
(43) Pub. Date: Jul. 21, 2016

(54) COMPUTER-IMPLEMENTED INTEGRATED HEALTH SYSTEMS AND METHODS

(71) Applicant: Soar BioDynamics, Ltd., Incline Village, NV (US)

(72) Inventors: Thomas B. Neville, Incline Village, NV (US); David Kaminski, Cambridge, MA (US)

(21) Appl. No.: 14/832,572

(22) Filed: Aug. 21, 2015

Related U.S. Application Data
(63) Continuation of application No. 13/454,058, filed on Apr. 23, 2012, now abandoned.
Application No. 13/454,058 is a continuation of application No. 11/581,226, filed on Oct. 13, 2006, now abandoned.
(60) Provisional application No. 60/726,514, filed on Oct. 13, 2005.

(51) Int. Cl. G01N 33/574; A61N 5/00; G06F 19/00; A61B 10/02

(52) U.S. Cl. CPC G01N 33/57434; A61B 10/02; A61N 5/00; G06F 19/366; G01N 2333/96433

(57) ABSTRACT

Computer-implemented integrated health systems and methods related to organs of the human body and to cancer. For example, a method and a system can be configured for choosing a strategy for an organ condition that maximizes a life score.

### CROSS-REFERENCE TO RELATED APPLICATIONS

This application claims priority to and the benefit of U.S. Provisional Application Ser. No. 60/726,514, (entitled “Computer-Implemented Prostate Cancer Treatment Systems And Methods” filed on Oct. 13, 2005), of which the entire disclosure (including any and all figures) is incorporated herein by reference. This application is related to the following co-owned U.S. patent applications: “Computer-Implemented Personal And Relationship Assessment Systems And Methods” (Ser. No. 11/431,248 and filed May 9, 2006); “Computer-Implemented Cancer Assessment Systems And Methods” (Ser. No. 11/431,119 and filed May 9, 2006); “Computer-Implemented Personal Analysis Methods And Systems” (Ser. No. 11/431,157 and filed May 9, 2006); and “Computer-Implemented Systems And Methods For Analyzing Medical Conditions” (Ser. No. 11/431,156 and filed May 9, 2006). The entire disclosures (including any and all figures) of all of the aforementioned patent applications are incorporated herein by reference.

### BACKGROUND

This document discloses computer-implemented integrated health systems and methods related to organs of the human body and to cancer.

In the field of medicine there is increasing emphasis on: health, disease prevention and early detection and treatment; avoiding unnecessary treatment; choosing the optimal timing of the best treatment based on medical evidence; and avoiding invasive and costly procedures like biopsies. The use of screening blood tests is also becoming more prevalent and cost effective. One blood draw reduces costs of screening for many conditions. New techniques reduce the cost of specific tests. The incremental cost of additional tests decreases once blood is drawn for another test. Blood can be stored for later testing if needed for specific conditions in order to reduce costs.

Another approach is periodic whole body imaging. Periodic whole body imaging is becoming more prevalent as part of a screening program and/or triggered by warning signs for one condition. Total costs are declining from $1,000, where the average cost per organ is less than $100. The Incremental cost for each additional organ is very low once imaging of other organs is initiated. Organ volume measurements and other image processing is becoming increasingly automated and is dropping in cost.

Significant investments are being made to accelerate discovery and use of biomarkers that effectively detect progressing cancer. However, many of the new biomarkers are not adequately effective based on the results of one test.

### SUMMARY

This document discloses computer-implemented integrated health systems and methods related to organs of the human body and to cancer.

For example, a method is disclosed for choosing a strategy for an organ condition that maximizes Life Score based on personalized estimates as a function of strategy of: the probability of the condition, for example that cancer is progressing, the severity of the condition, for example the amount of early warning, and the Cure Ratio.

Another method is disclosed for choosing the timing for treatment that maximizes Life Score based on personalized estimates of the probability of the condition, for example that cancer is progressing, and the Cure Ratio as a function of timing.

A method is disclosed for estimating trends over time for test results of two biomarkers and their ratio where pairs of test results are excluded from trend estimation if they fall outside an acceptable tolerance area, or oval, around the trend at the time of the tests where the tolerance area, or oval, is measured either: In terms of the two variables, or in terms of one variable and the ratio of one variable to the other. A related version of the method is also disclosed for estimating a trend over time for test results of a biomarker where some test results are excluded from trend estimation if they fall outside an acceptable tolerance range around the trend at the time of the tests. Another related version of the method is disclosed for estimating trends over time for test results of two biomarkers and their ratio.

A method is disclosed for estimating trends in residual velocities over time for a biomarker by one of two equivalent methods. In one method (Velocity Calculation Method), trend velocities are calculated as the annual rate of change of the biomarker trend at any time, and trend velocities in the absence of progressing cancer are predicted based on information that may include: one or more values of the biomarker trend, one or more velocities of the biomarker trend, one or more measured values of a secondary variable such as volume of the organ, one or more estimated trend values of a secondary variable, or one or more velocities of the trend for the secondary variable. Residual velocities are calculated by subtracting predicted velocities from trend velocities. In the other method (Trend Calculation Method), the biomarker trend is estimated; the trend in the absence of progressing cancer is predicted based on information that may include: one or more values of the biomarker trend, one or more velocities of the biomarker trend, one or more measured values of a secondary variable such as volume of the organ, one or more estimated trend values of a secondary variable, or one or more velocities of the trend for the secondary variable. The residual trend is calculated by subtracting the predicted trend from the trend. Residual velocities are calculated as the annual rate of change in the residual trend. A related version of the method is also disclosed for estimating trends in velocities over time for a biomarker by calculating the annual rate of change in the estimated trend.

A method is disclosed for estimating the severity of one or more conditions of an organ, including: the years of early warning before the cure rate for progressing cancer begins to decline steeply where the residual velocity of a biomarker is mapped to years of early warning by comparing the residual velocity with a typical residual velocity trend of that marker for progressing cancer vs years of early warning; the severity of temporary conditions, such as an infection; and the severity of long-term conditions, such as the amount of organ volume growth.

A method is disclosed for determining the alert level for progressing cancer by comparing the residual velocity trend for one biomarker plus either the residual velocity trend for a second biomarker or the ratio of the second to the first residual velocity trend with a two dimensional map of alert levels.

A method is disclosed for estimating the probability of one or more conditions of an organ, including: First, cancer is progressing based on: prior probabilities of a range of years of early warning of progressing cancer based on personal risk factors for the individual considered; a probability distribution for no progressing cancer around the predicted values for the trend residual velocity for one biomarker and either the trend residual velocity for a second biomarker or the ratio of the second to the first trend residual velocity where both biologic uncertainty and trend uncertainty are taken into account; and a probability distributions for one or more years of early warning of progressing cancer, based on population studies, for the trend residual velocity for one biomarker and either the trend residual velocity for a second biomarker or the ratio of the second to the first trend residual velocity where both biologic uncertainty and trend uncertainty are taken into account; Second, temporary conditions, such as an infection; and Third, long-term conditions, such as organ volume growth.

A method is disclosed for screening for progressing cancer and other conditions of an organ that consists of: First, estimating trends over time for test results of two biomarkers and their ratio where pairs of test results are excluded from trend estimation if they fall outside an acceptable tolerance area, or oval, around the trend at the time of the tests where the tolerance area, or oval; is measured either: In terms of the two variables, or in terms of one variable and the ratio of one variable to the other; Second, estimating trends in residual velocities over time for two biomarkers by one of two equivalent methods: a Velocity Calculation Method or a Trend Calculation Method; Third, estimating the severity of one or more conditions of an organ, including: the years of early warning before the cure rate for progressing cancer begins to decline steeply where the residual velocity of a biomarker is mapped to years of early warning by comparing the residual velocity with a typical residual velocity trend of that marker for progressing cancer vs years of early warning; the severity of temporary conditions, such as an infection; and the severity of long-term conditions, such as the amount of organ volume growth; Fourth; determining the alert level for progressing cancer by comparing the residual velocity trend for one biomarker plus either the residual velocity trend for a second biomarker or the ratio of the second to the first residual velocity trend with a two dimensional map of alert level; and Fifth, A method is disclosed for estimating the probability of one or more conditions of an organ, including: 1) cancer is progressing based on: prior probabilities of a range of years of early warning of progressing cancer based on personal risk factors for the individual considered; a probability distribution for no progressing cancer around the predicted values for the trend residual velocity for one biomarker and either the trend residual velocity for a second biomarker or the ratio of the second to the first trend residual velocity where both biologic uncertainty and trend uncertainty are taken into account: and a probability distributions for one or more years of early warning of progressing cancer, based on population studies, for the trend residual velocity for one biomarker and either the trend residual velocity for a second biomarker or the ratio of the second to the first trend residual velocity where both biologic uncertainty and trend uncertainty are taken into account; 2) temporary conditions, such as an infection; and 3) long-term conditions, such as organ volume growth.

Another method is disclosed for screening for progressing cancer and other conditions of an organ that consists of: First, estimating trends over time for test results of two biomarkers and their ratio; Second, estimating trends in residual velocities over time for two biomarkers by one of two equivalent methods: a Velocity Calculation Method or a Trend Calculation Method; Third, estimating the severity of one or more conditions of an organ, including: the years of early warning before the cure rate for progressing cancer begins to decline steeply where the residual velocity of a biomarker is mapped to years of early warning by comparing the residual velocity with a typical residual velocity trend of that marker for progressing cancer vs years of early warning; the severity of temporary conditions, such as an infection, and the severity of long-term conditions, such as the amount of organ volume growth; Fourth, determining the alert level for progressing cancer by comparing the residual velocity trend for one biomarker plus either the residual velocity trend for a second biomarker or the ratio of the second to the first residual velocity trend with a two dimensional map of alert level; and Fifth, A method is disclosed for estimating the probability of one or more conditions of an organ, including: 1) cancer is progressing based on: prior probabilities of a range of years of early warning of progressing cancer based on personal risk factors for the individual considered; a probability distribution for no progressing cancer around the predicted values for the trend residual velocity for one biomarker and either the trend residual velocity for a second biomarker or the ratio of the second to the first trend residual velocity where both biologic uncertainty and trend uncertainty are taken into account; and a probability distributions for one or more years of early warning of progressing cancer, based on population studies, for the trend residual velocity for one biomarker and either the trend residual velocity for a second biomarker or the ratio of the second to the first trend residual velocity where both biologic uncertainty and trend uncertainty are taken into account; 2) temporary conditions, such as an infection; and 3) long-term conditions, such as organ volume growth.

Another method is disclosed for screening for progressing cancer and other conditions of an organ that consists of: First, estimating a trend over time for test results of a biomarker where some test results are excluded from trend estimation if they fall outside an acceptable tolerance range around the trend at the time of the tests; Second, estimating trends in residual velocities over time for a biomarker by one of two equivalent methods: a Velocity Calculation Method or a Trend Calculation Method; Third, estimating the severity of one or more conditions of an organ, including: the years of early warning before the cure rate for progressing cancer begins to decline steeply where the residual velocity of a biomarker is mapped to years of early warning by comparing the residual velocity with a typical residual velocity trend of that marker for progressing cancer vs years of early warning; the severity of temporary conditions, such as an infection; and the severity of long-term conditions, such as the amount of organ volume growth; Fourth, determining the alert level for progressing cancer by comparing the residual velocity trend for a biomarker with a one dimensional map of alert level; and Fifth, A method is disclosed for estimating the probability of one or more conditions of an organ, including: 1) cancer is progressing based on: prior probabilities of a range of years of early warning of progressing cancer based on personal risk factors for the individual considered; a probability distribution for no progressing cancer around the predicted values for the trend residual velocity for a biomarker where both biologic uncertainty and trend uncertainty are taken into account; and probability distributions for one or more years of early warning of progressing cancer, based on population studies, for the trend residual velocity for one biomarker where both biologic uncertainty and trend uncertainty are taken into account; 2) temporary conditions, such as an infection; and 3) long-term conditions, such as organ volume growth.

A method is disclosed for screening for progressing cancer and other conditions of an organ that consists of: First, estimating trends over time for test results of two biomarkers and their ratio where pairs of test results are excluded from trend estimation if they fall outside an acceptable tolerance area, or oval, around the trend at the time of the tests where the tolerance area, or oval, is measured either: In terms of the two variables, or in terms of one variable and the ratio of one variable to the other; Second, estimating trends in velocities over time for two biomarkers by calculating the annual rates of change in their estimated trends; Third, estimating the severity of one or more conditions of an organ, including the years of early warning before the cure rate for progressing cancer begins to decline steeply where the velocity of a biomarker is mapped to years of early warning by comparing the velocity with a typical velocity trend of that marker for progressing cancer vs years of early warning; the severity of temporary conditions, such as an infection; and the severity of long-term conditions, such as the amount of organ volume growth; Fourth, determining the alert level for progressing cancer by comparing the velocity trend for one biomarker plus either the velocity trend for a second biomarker or the ratio of the second to the first velocity trend with a two dimensional map of alert level; and Fifth, A method is disclosed for estimating the probability of one or more conditions of an organ, including: 1) cancer is progressing based on: prior probabilities of a range of years of early warning of progressing cancer based on personal risk factors for the individual considered; a probability distribution for no progressing cancer around the predicted values for the trend velocity for one biomarker and either the trend velocity for a second biomarker or the ratio of the second to the first trend velocity where both biologic uncertainty and trend uncertainty are taken into account; and a probability distributions for one or more years of early warning of progressing cancer, based on population studies, for the trend velocity for one biomarker and either the trend velocity for a second biomarker or the ratio of the second to the first trend velocity where both biologic uncertainty and trend uncertainty are taken into account; 2) temporary conditions, such as an infection; and 3) long-term conditions, such as organ volume growth.

A method is disclosed for screening for progressing cancer and other conditions of an organ that consists of: First, estimating trends over time for test results of two biomarkers and their ratio where pairs of test results are excluded from trend estimation if they fall outside an acceptable tolerance area, or oval, around the trend at the time of the tests where the tolerance area, or oval, is measured either: In terms of the two variables, or in terms of one variable and the ratio of one variable to the other; Second, estimating trends in residual velocities over time for two biomarkers by one of two equivalent methods: a Velocity Calculation Method or a Trend Calculation Method; Third, estimating the severity of one or more conditions of an organ, including: the years of early warning before the cure rate for progressing cancer begins to decline steeply where the residual velocity of a biomarker is mapped to years of early warning by comparing the residual velocity with a typical residual velocity trend of that marker for progressing cancer vs years of early warning; the severity of temporary conditions, such as an infection; and the severity of long-term conditions, such as the amount of organ volume growth; Fourth, determining the alert level for progressing cancer by comparing the residual velocity trend for one biomarker plus either the residual velocity trend for a second biomarker or the ratio of the second to the first residual velocity trend with a two dimensional map of alert level.

A method is disclosed for screening for progressing cancer and other conditions of an organ that consists of: First, estimating trends over time for test results of two biomarkers and their ratio where pairs of test results are excluded from trend estimation if they fall outside an acceptable tolerance area, or oval, around the trend at the time of the tests where the tolerance area, or oval, is measured either: In terms of the two variables, or in terms of one variable and the ratio of one variable to the other; Second, estimating trends in residual velocities over time for two biomarkers by one of two equivalent methods: a Velocity Calculation Method or a Trend Calculation Method; Third, estimating the severity of one or more conditions of an organ, including: the years of early warning before the cure rate for progressing cancer begins to decline steeply where the residual velocity of a biomarker is mapped to years of early warning by comparing the residual velocity with a typical residual velocity trend of that marker for progressing cancer vs years of early warning; the severity of temporary conditions, such as an infection; and the severity of long-term conditions, such as the amount of organ volume growth.

A method is disclosed for screening for progressing cancer and other conditions of an organ that consists of: First, estimating trends over time for test results of two biomarkers and their ratio where pairs of test results are excluded from trend estimation if they fall outside an acceptable tolerance area, or oval, around the trend at the time of the tests where the tolerance area, or oval, is measured either: In terms of the two variables, or in terms of one variable and the ratio of one variable to the other; and Second, estimating trends in residual velocities over time for two biomarkers by one of two equivalent methods: a Velocity Calculation Method or a Trend Calculation Method.

Another method is disclosed for improving the ability of the invention to estimate the probability of progression using feedback learning from the results of analysis of progression and other variables for more than one man.

A method is disclosed for improving the ability of the invention to estimate the Cure Ratio and cure rates using feedback learning from the results of analysis of cancer recurrence and other variables for more than one man.

Another method is disclosed for improving the effectiveness of the invention using feedback learning where the experience of more than one man with enlarging prostates is analyzed in order to improve the predictions of PSA, Free PSA and other test results as a function of prostate volume and other variables and to estimate probability distributions for those predictions.

A method is disclosed for improving the effectiveness of the invention using feedback learning where the experience of more than one man with infections is analyzed in order to improve the use of PSA, Free PSA and other test results and their residual velocities to identify test results distorted by infections.

Another method is disclosed for improving the effectiveness of the invention using feedback learning where the experience of more than one man who have changed medication or made other changes is analyzed in order to improve the predictions of PSA, Free PSA and other test results as a function of the changes and to estimate probability distributions for those predictions.

A method is disclosed for improving the effectiveness of the invention using feedback learning where the experience of more than one man with progressing prostate cancer is analyzed in order to improve the use of PSA, Free PSA and other test results and their residual velocities to identify progressing prostate cancer and to estimate probability distributions for those variables.

### BRIEF DESCRIPTION OF THE DRAWINGS

FIG. 1 is a block diagram depicting an integrated health system.

FIG. 2 is a block diagram depicting an integrated organ health system.

FIG. 3 is a graph depicting confined and penetrating progression.

FIG. 4 is a graph depicting a cure ratio graph.

FIG. 5 is flowchart depicting a cure ratio calculation method.

FIG. 6 depicts processing of Pretreatment PSA, Gleason and stage information.

FIG. 7 is a table depicting example cancer score values.

FIG. 8 depicts example cancer score tables.

FIG. 9 depicts an example for calculating an average cancer score.

FIG. 10 is a flowchart depicting a strategy system flowchart.

FIG. 11 depicts an example for estimating life scores for treatment timing.

FIGS. 12 and 13 are graphs for treatment strategies.

FIG. 14 is a flowchart depicting a treatment timing system flowchart.

FIG. 15 is a flowchart for estimating life score for treatment timing.

FIG. 16 is a life score graph.

FIG. 17 is a life score impact graph.

FIG. 18 depicts an example of a dynamic screening system.

FIG. 19 is a flowchart for a dynamic screening system.

FIG. 20 is a flowchart for trends processing.

FIG. 21 is a flowchart for calculating velocity distributions.

FIG. 22 depicts estimation for multiple volume measurements.

FIG. 23 depicts estimation for one volume measurements.

FIG. 24 depicts estimation for no volume measurement.

FIGS. 25-28 are flowcharts for prediction generation.

FIG. 29 is a flowchart depicting a method for mapping residual results.

FIG. 30 is a flowchart depicting a method for predicting PSA and free PSA.

FIG. 31 depicts hypothesis generation for progressing cancer.

FIG. 32 depicts prior probabilities processing.

FIG. 33 depicts long-term probabilities processing.

FIG. 34 is a flowchart for calculating probability of progressing cancer.

FIGS. 35 and 36 are flowcharts for estimating the probability of progressing cancer.

FIG. 37 depicts a dynamic screening custom content system.

FIGS. 38-46 depict feedback processing scenarios.

FIGS. 47 and 48 are block diagrams depicting examples of integrated health systems.

### DETAILED DESCRIPTION

### I. Integrated Health System

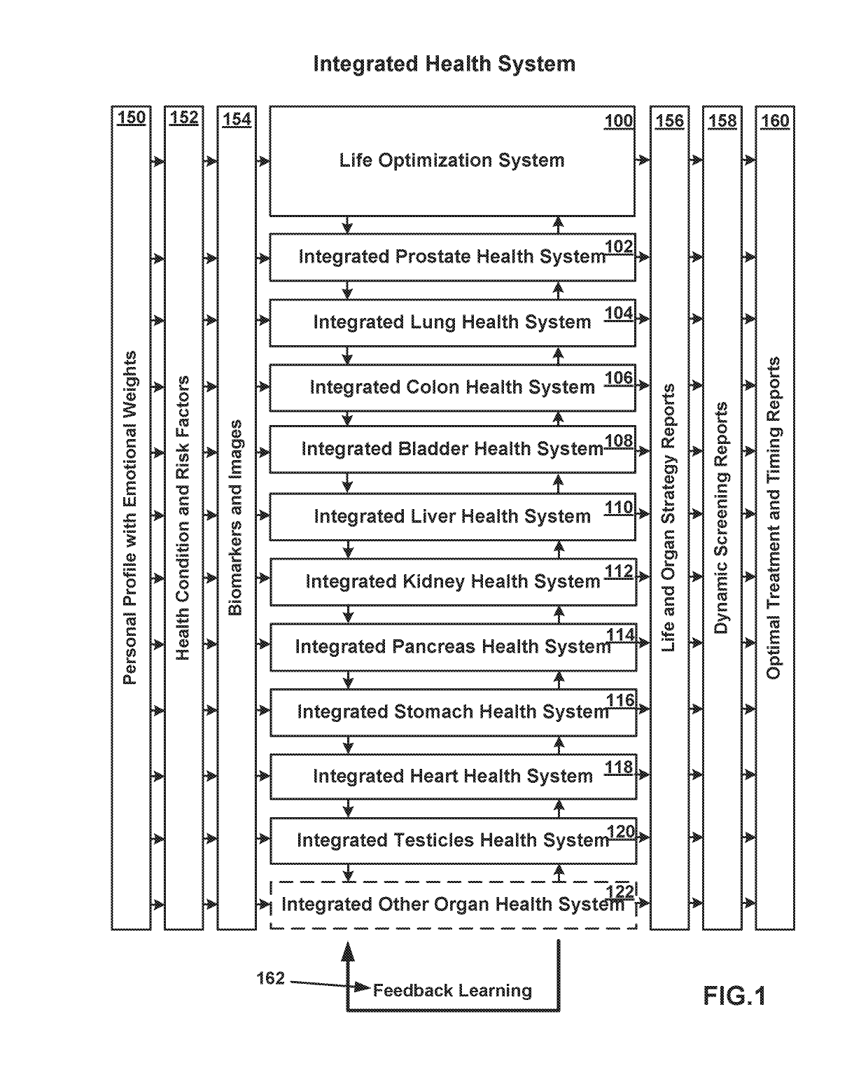

Integrated Health Systems, shown on FIG. 1, combine at least two subsystems that may include the Life Optimization System (100) and one or more Integrated Organ Health Systems (102 through 122). Inputs may include: a person's personal Profile with emotional weights (150), health conditions and risk factors (152), and the results of biomarker tests and analysis of images of the body and some of its organs (154). Outputs may include: life and organ strategy reports (156), Dynamic Screening reports (158), and optimal treatment and timing reports (160). Feedback learning from actual results improves the effectiveness of the systems (162).

### Integrated Organ Health Systems

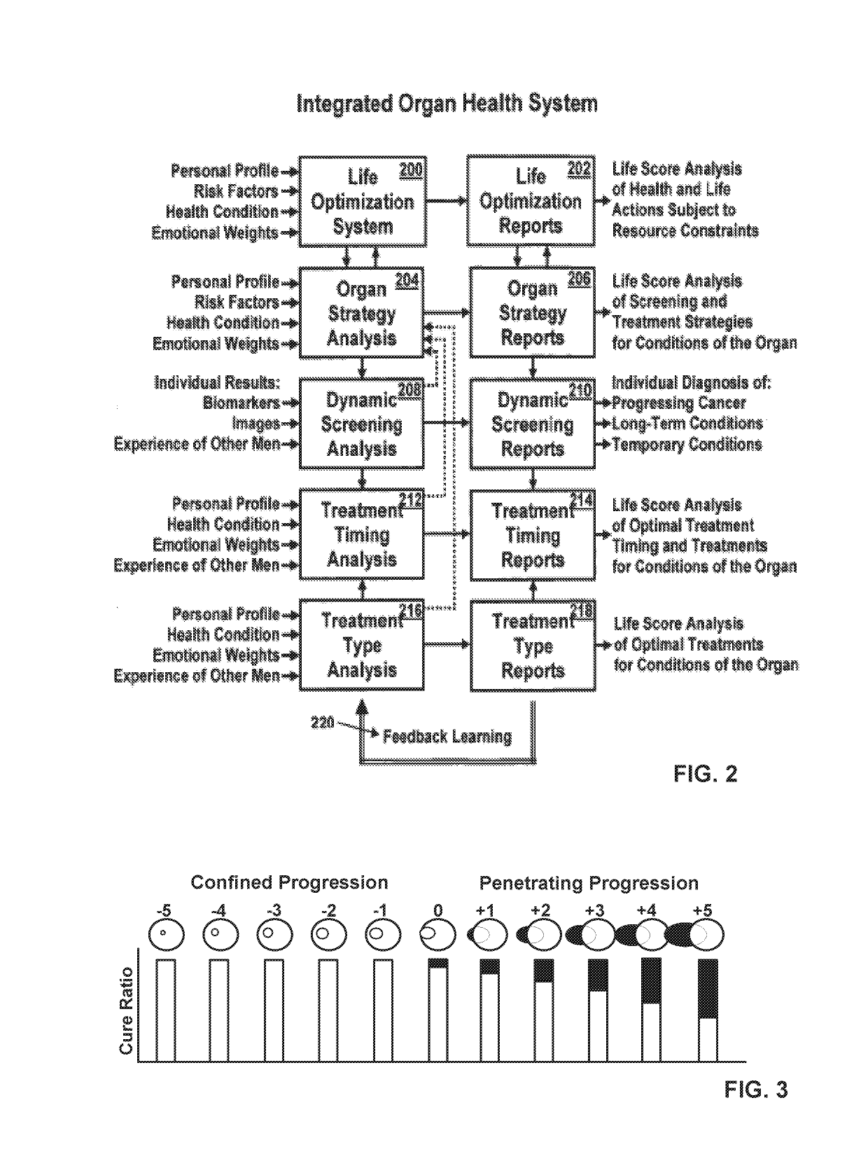

Integrated Organ Health Systems, shown on FIG. 2, comprise analysis and report systems for Organ Strategy (204 and 205), Dynamic Screening (208 and 210), Treatment Timing and Treatment Type (200 and 202). They may connect with the Life Optimization System (200 and 202), for which a patent application has been submitted. The Organ Strategy and Treatment Timing Systems build on the Treatment Type Systems (216 and 218), for which a patent application has been submitted. All systems will improve over time using feedback learning (220) from the experience of users of the systems.

### Prostates and Prostate Cancer

Integrated Organ Health Systems apply to a wide range of human organs, as suggested by the Integrated Health System chart above. The rest of the application will focus on the male prostate and prostate cancer as a concrete example that does not limit the generality of the invention.

### II. Progressing Cancer and the Cure Ratio

The Cure Ratio is an element of the Organ Strategy System and the Treatment Timing System when applied to cancer. It focuses on curable internal progressing cancer because it is the only prostate cancer that needs to be treated and can be cured.

### Four Prostate Cancer States

We consider four prostate cancer states: No Cancer, Dormant Cancer, Internal Progressing Cancer, and External Progressing Cancer. We define these states based on the probability they will occur because we are chiefly concerned with risk analysis rather than the complex biology of prostate cancer. We provide example illustrations to help you visualize the biology behind the probabilities, but caution you against reading too much into them. The risks you face, not the actual biological mechanisms, are what matter most for our analysis and your decisions.

No Cancer means absolutely no prostate cancer exists in your prostate or the surrounding areas. Cancer is neither inside your prostate nor outside it in this state.

Dormant Cancer is any prostate cancer that is not clearly progressing toward metastasis. The first Dormant Cancer cells start with a genetic mutation. These cells will remain dormant unless additional mutations are triggered. As long as cancer remains dormant, it poses no health or survival threat. Dormant Cancer can exist solely inside your prostate or outside as well as inside your prostate.

Internal Progressing Cancer is confined to the prostate. Additional mutations of Dormant Cancer cells can trigger progression. Progressing Cancer grows exponentially and will eventually lead to death unless treated. Treatment of the prostate removes or kills the growing prostate cancer and provides a full cure as long as any cancer outside the prostate remains dormant.

External Progressing Cancer is Progressing Cancer that exists outside of the prostate. Tiny amounts of Dormant Cancer that may already exist outside the prostate can start to progress, or Progressing Cancer cells in the prostate may find their way out. In either case, External Progressing Cancer is usually undetectable in its early stages. We know it exists in many men because of the significant amount of cancer recurrence after surgery that removes all of the cancer confined to the prostate. Undetectable cancer exists outside the prostate in these cases. External Progressing Cancer has a very high probability of being incurable.

### Curable Internal Progressing Cancer

In the following analysis, we focus on Internal Progressing Cancer because it can be cured. We exclude in this example both Dormant Cancer, which needs no treatment, and External Progressing Cancer, which usually cannot be cured. Internal progressing cancer is divided into two phases: the Confined Progression Phase and the Penetrating Progression Phase, as illustrated by the figure.

The Confined Progression Phase starts when the first Dormant Cancer cell mutates beyond its current state and begins exponential cancer cell multiplication. The graph on FIG. 3 begins five years before (−5) the Transition Point (at year 0) to the Penetrating Progression Phase. The tiny tumor on the left grows exponentially in total volume each year. During this phase, the cancer registers a Gleason score of 6 or less, and is labeled T1 stage cancer if detected by biopsy. The light bars suggest that the Cure Ratio is close to 100% on the left half of the graph, where the probability is high that cancer is confined to the prostate. The Cure Ratio is defined as the actual cure rate for Internal Progressing Cancer at a specific point in time divided by the maximum cure rate if treated very early in the progression process. The Cure Ratio does not apply to the case of Dormant Cancer, which is harmless, or External Progressing Cancer, which cannot be cured.

Transition Point—We define year zero in the middle of FIG. 3 as the Transition Point when the Cure Ratio starts to decline rapidly because of the increasing chance that cancer has penetrated the prostate and is growing outside. The Transition Point marks the change in slope in the Cure Ratio rather than any specific biological state. The light bar at year zero is slightly less than 100%, which means that not all Internal Progressing Cancer can be cured. The thin dark bar above it corresponds to the small risk that cancer has penetrated the prostate and is no longer curable.

Penetrating Progression Phase—The positive numbers on the right side of the graph on FIG. 3 show the number of years into the Penetrating Progression Phase. This phase is characterized by the increasing risk that prostate cancer has penetrated beyond the prostate, shown by the dark ovals and the corresponding decreasing Cure Ratio. The shorter light bars on the right half of the graph indicate that the Cure Ratio drops steeply each year. The increasingly tall dark bars indicate the growing probability each year that cancer has penetrated beyond the prostate and is no longer curable. The diagram shows the dark cancer penetrating beyond the prostate in order to indicate the increasing risk that penetration has occurred. For example, at year+3 there is a roughly 25% chance that cancer has penetrated far enough to become incurable and a 75% chance that cancer has not penetrated and is still curable.

The Cure Ratio Declines with Progression

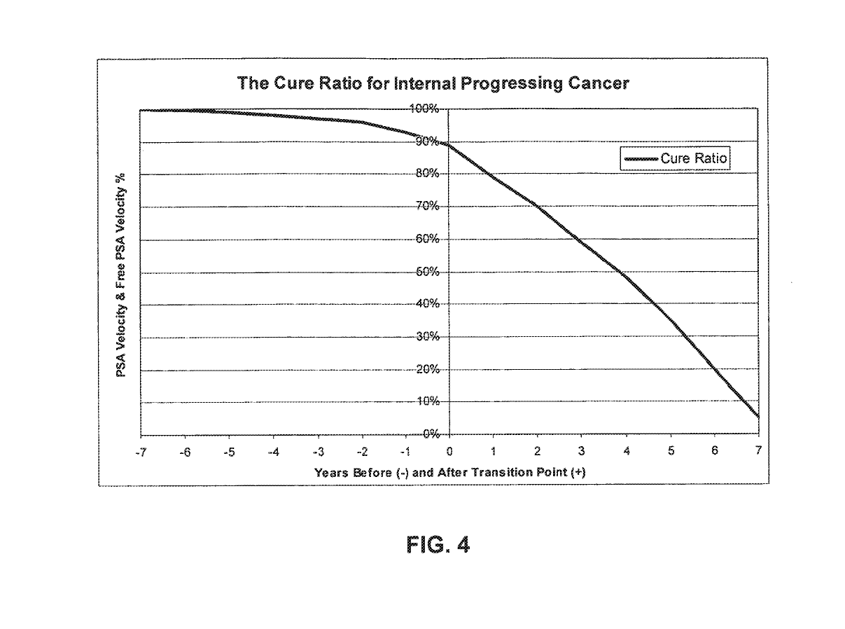

The Cure Ratio graph on FIG. 4 shows our estimate of the Cure Ratio for Internal Progressing Cancer. Early Treatment Has a Small Cure Ratio Benefit. The curve is relatively flat during the Confined Progression Phase on the left side of the graph. Therefore, treatment a year earlier at any point in this phase provides only a small benefit in terms of increased Cure Ratio. Late Treatment Has a High Cure Ratio Cost. The curve drops steeply during the Penetrating Progression Phase on the right side of the graph. Therefore, treatment a year later at any point in this phase imposes a high cost in terms of reduced Cure Ratio.

### Cure Ratio Calculation

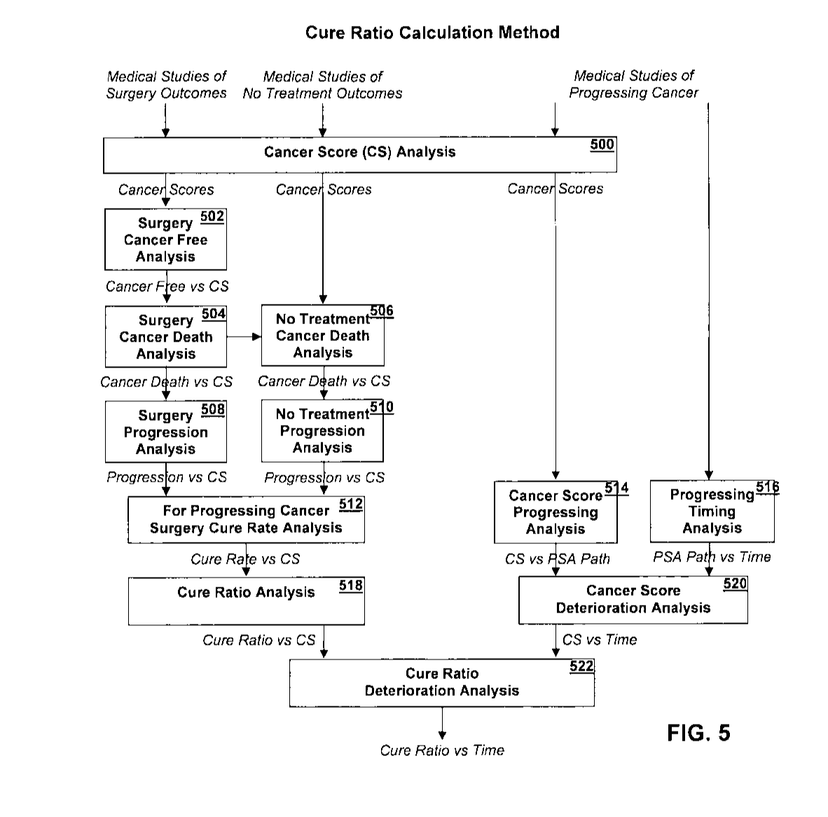

The Cure Ratio calculation is shown by the flow chart on FIG. 5.

### Cancer Score Analysis

Cancer Score Analysis is carried out in the top module (500) on FIG. 5. Cancer Score (CS) is our summary measure of how favorable or unfavorable a particular cancer is. We use it to compare results on a consistent basis and to quantify trends more rigorously than is possible using discrete groups (of ranges of PSA, Gleason, Stage or combinations).

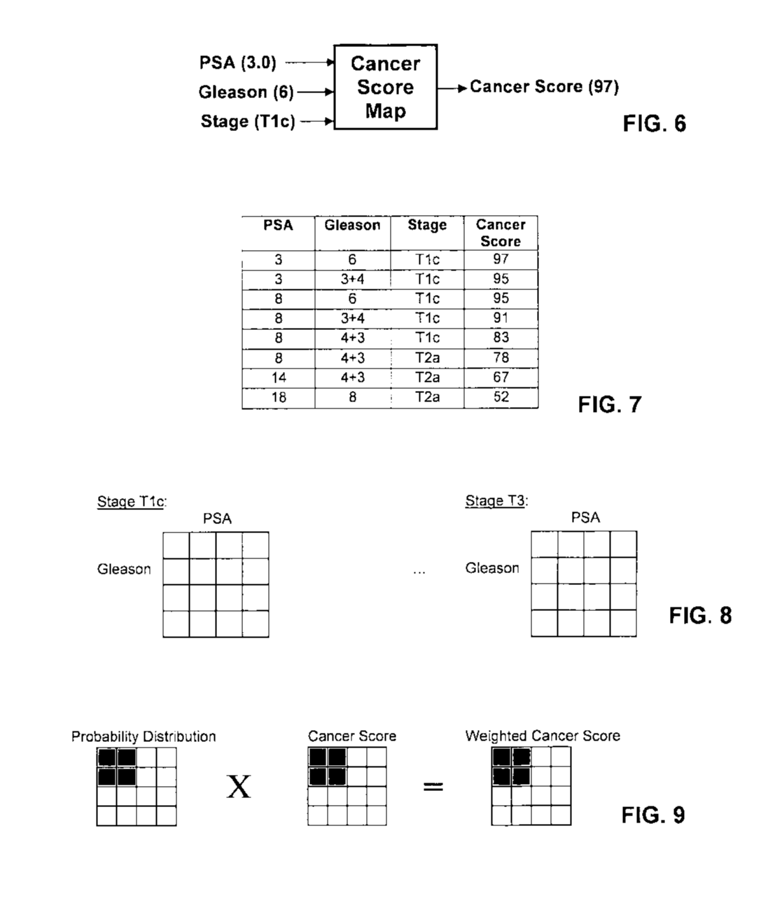

Cancer Score is a single measure of prostate cancer condition. Pretreatment PSA. Gleason and Stage are transformed into a single Cancer Score, as shown in the schematic on FIG. 6.

We base our Cancer Score on the projected ten year Johns Hopkins results and have confirmed its validity and usefulness on results from the Cleveland Clinic. Some example Cancer Score values for representative combinations of PSA, Gleason and Stage are shown on FIG. 7.

Cancer Scores are available for a wide range of PSA and Gleason values for Stages T1c through T3—just like the Partin tables. These Cancer Score tables are represented schematically on FIG. 8—with one table for each Stage.

Cancer Score is useful because it allows us to compare the results of different studies on a consistent basis. Here are a few of the ways populations are grouped for analysis: Favorable and Unfavorable cancer—defined in different ways; Favorable, Intermediate and Unfavorable cancer—defined in different ways; and Grouped by cancer variable: PSA—sometimes grouped in different ways, Gleason—sometimes grouped in different ways, and Stage—sometimes grouped in different ways.

Without Cancer Score, studies using one set of groups can't be compared directly to studies using any other set of groups. Even if two studies use the same definitions of groups the results may not be comparable. To be comparable they need both the same definition and the same distribution of PSA, Gleason and Stage in each of the equivalent groups. For example, two Favorable groups defined the same way may have significantly different average Cancer Scores because one Favorable Group has a high concentration of very early stage cancer with low PSA, Gleason and Stage—while the other group has a high concentration of poorer cancer that just squeaks above the definition of Favorable. These studies are not comparable even though they use the same definitions of groups.

Cancer Score solves one, more or all these problems. We can plot results using all these groups on a consistent basis using Cancer Score. A step is to calculate the average Cancer Score for each group in a study. The process for calculating average Cancer Score may use the following steps, as suggested by FIG. 9:
1. Create probability distribution table for PSA. Gleason and Stage.
2. Highlight combinations that define the group in both the Cancer Score and distribution tables (for example, the green cells).
3. Multiply each individual probability by its corresponding Cancer Score.
4. Sum the highlighted probability weighted Cancer Scores.
5. Divide the total weighted Cancer Scores by the total probability for the highlighted cells to get the average Cancer Score for the group.

Fortunately, most articles report in Table 1 at least the distributions of PSA, Gleason and Stage alone. Some studies provide more details of the joint distributions. This information allows us to estimate the joint probability distribution (step 1) to complete this process.

### Surgery Analysis

The goal of surgery analysis is to estimate the time path of progression after surgery that is comparable to no treatment for the full range of Cancer Scores. Surgery is chosen as the reference treatment for two reasons: no other treatment has proven to have better cure rates and detection of recurrence is prompt and unambiguous.

Cancer Score Analysis is performed in module 500 on FIG. 5. Medical studies of surgery outcomes are obtained and Cancer Scores are estimated for each analysis group in the study population, including the whole population.

Cancer Free Analysis is performed in module 502 on FIG. 5. Medical journal articles report biochemical freedom from cancer recurrence vs years after treatment for a variety of different groups. Fortunately, there is reasonably close agreement reported among the top doctors with long time series. Response surfaces are estimated using a number of studies for freedom from cancer recurrence after surgery as a function of time after treatment and Cancer Score.

Cancer Death Analysis is performed in module 504 on FIG. 5. Response surfaces are estimated using the results of the cancer free analysis combined with studies of cancer death following recurrence combined with long term studies of death from cancer after surgery. Johns Hopkins has done the most extensive long-term analysis of death following recurrence after surgery. The probability of cancer death is estimated as a function of time after surgery and Cancer Score.

Progression Analysis is performed in module 508 on FIG. 5. Response surfaces are estimated using the results of the cancer death analysis and cancer free analysis for progression after recurrence as a function of time and Cancer Score that is consistent with progression for no treatment After an initial transition period, detectable progression (recurrence) seems to precede cancer death by roughly fifteen years on average

### No Treatment Analysis

The goal of no treatment analysis is to estimate the time path of detectable progression for no treatment that is comparable to surgery for the full range of Cancer Scores.

Cancer Score Analysis is performed in module 500 on FIG. 5. Medical studies of no treatment outcomes are obtained and Cancer Scores are estimated for each analysis group in the study population, including the whole population.

Cancer Death Analysis is performed in module 506 on FIG. 5. Our analysis is informed by a wide variety of medical studies (e.g., two landmark studies to define the relationship between no treatment and surgery results). We start with the surgery results because they provide the most detail over time and Cancer Score. We consider the relationship between surgery and no treatment found in an ongoing randomized trial reported in the New England Journal of Medicine. Surgery was better but not by an enormous amount. We also consider the excellent long-term outcomes for men with very favorable cancer reported in articles by Albertsen, the most recent reported in the NEJM. It appears clear that long-term no treatment outcomes converge toward surgery for very favorable cancer. Response surfaces are estimated relative to surgery using these and other results. The probability of cancer death is estimated as a function of time after diagnosis and Cancer Score.

Progression Analysis is performed in module 510 on FIG. 5. Response surfaces are estimated using the results of the cancer death analysis and estimates of the lag from detectable progression to cancer death as a function of time and Cancer Score that is consistent with progression for surgery. After an initial transition period, detectable progression seems to precede cancer death by roughly fifteen years on average

### Cure Ratio Vs Cancer Score

A goal can be to estimate for progressing cancer the time path of Cure Ratio as a function of Cancer Score at diagnosis.

For Progressing Cancer Surgery Cure Rate Analysis is performed in module 512 on FIG. 5. The probability of progressing cancer is calculated from the results of the no treatment progression analysis. The cure rate conditional on cancer progressing is calculated using this result combined with the overall cure rate of surgery for all cancer from the surgery progression analysis step. The result is an estimate of the response surface for progressing cancer cure rate as a function of time after surgery and Cancer Score.

This probability can be expressed as the sum of the probability of no progression and the probability of cure after progression:

PrNCDbp@30=PrNCDbp@30(nt)+PrProg@30*PrNCDap@30(no cancer death)(no progression)+(cure after progression)
Where:
PrNCDbp@30: Prob of no cancer death at 30 before progression
PrNCDbp@30(nt): Prob of no cancer death at 30 before progression for for no treatment
PrProg@30: Prob of progression at 30
PrNCDap@30: Prob no cancer death at 30 after progression
   No Treatment is an example of this equation:

PrNCDbp@30=PrNCDbp@30(nt)+PrProg@30*PrNCDap@30(no cancer death)(no progression)+(cure)73%=73%+27%*0%

If cancer progresses no treatment lets it continue to progress—there is no cure from no treatment.
Surgery Now has some chance of curing cancer even though it will progress with no treatment—that is why men choose surgery. The surgery version of the equation for a Cancer Score of 95 looks like this:

PrNCDbp@30=PrNCDbp@30(nt)+PrProg@30+PrNCDap@30(no cancer death)(no progression)+(cure)84%=73%=27%*40%

Cure Ratio Analysis is performed in module 518 on FIG. 5. The Cure Ratio is the normalized cure rate with the cure rate for very favorable cancer defined as 100%. The Cure Ratio is an increasing function of Cancer Score—on the downside lower Cancer Scores lead to lower Cure Ratios.

### Progressing Cancer Analysis

The goal is to estimate for internal progressing cancer the time path of Cancer Score from very early stage cancer to incurable.

Progressing Timing Analysis is performed in module 516 on FIG. 5. The PSA path of progressing cancer versus time is estimated from medical studies of the natural history of progressing cancer. A good article is by Berger. It shows PSA trajectories vs year of detection for progressing cancer for three groups. One group was detected early with Gleason 6—favorable cancer. The second group was detected later with Gleason 7—intermediate cancer. The third group was detected even later with Gleason 8-10—unfavorable cancer. The average PSA at detection was lowest for favorable cancer and highest for unfavorable cancer as you would expect. We shifted the individual curves in time to form a continuous and consistent PSA path for progressing cancer independent of what stage it was detected.

Cancer Score Analysis is performed in module 500 on FIG. 5. Cancer Scores are estimated for each of the three Gleason detections groups, as shown on the previous graphs.

Cancer Score Progressing Analysis is performed in module 514 on FIG. 5 Cancer Scores are related to the PSA path for progressing cancer.

Cancer Score Deterioration Analysis is performed in module 520 on FIG. 5. Cancer Scores are plotted vs time for the three detection groups. Detection of Gleason 6 remains the reference year (0). We estimate that Cancer Score slowly approaches 100 in prior years, to the left. We project continued roughly linear decline in Cancer Score as cancer progresses.

### Cure Ratio for Progressing Cancer

The goal is to estimate for internal progressing cancer the deterioration in Cure Ratio over time, relative to the Transition Point.

Cure Ratio Deterioration Analysis is performed in module 522 on FIG. 5. The results of the Cure Ratio analysis and the progressing cancer analysis are combined to estimate the deterioration in Cure Ratio over time for progressing cancer. We have Cure Ratio as a function of Cancer Score and Cancer Score as a function of time. Together the allow us to plot Cure Ratio as a function of time.

### III. Organ Strategy System

Male prostates are subject to a variety of conditions, such as: Infection—a temporary condition; Volume growth caused by Benign Prostatic Hyperplasia (BPH)—a long-term condition; and Progressing prostate cancer which is distinct from dormant prostate cancer. Prostate cancer is the focus of this organ strategy system example because of its importance and the level of medical controversy surrounding it. Similar strategic analysis can be applied to other conditions of the prostate, and of course to other organs.

### Competing Prostate Cancer Strategies

Doctors strongly disagree about the best prostate cancer strategy. Urologists and other prostate cancer specialists usually recommend a Cancer Dominated Strategy that emphasizes aggressive screening and immediate treatment of prostate cancer. Other doctors, who are more focused on preventive medicine, oppose screening, or do not recommend it, because they believe it leads to unnecessary treatment and side effects. We call this a Treatment Avoidance Strategy. We encourage consideration of a third strategy—the Best Life Strategy. This strategy leads to an optimal combination of screening and treatment that offers both a long life and a high level of well-being.

The American Urology Association and the American Cancer Society recommend PSA screening and, implicitly, the aggressive detection and immediate treatment of prostate cancer. Our analysis shows that over their lifetime men have a very high probability of dormant cancer that will not harm them and a low probability of progressing cancer that might. The harder doctors look for prostate cancer the more likely they are to find the dormant cancer that probably exists in the prostate. When focused on prostate cancer the best and most well intentioned medical care increases the chance of finding and unnecessarily treating dormant cancer. The result is an excessive risk of impotence, incontinence, and other side effects of unnecessary primary treatment—surgery and different types of radiation. As screening methods improve, many doctors are moving closer to finding and treating all detectable prostate cancer, whether dormant or not. Ironically, the risk of unnecessary side effects increases as screening and detection methods improve. Therefore, the best cancer dominated medical care may not be best.

The American College of Physicians, the American College of Preventive Medicine and the U.S. Preventative Services Task Force either oppose PSA screening, or do not recommend it, because it is associated with a high risk of over-treatment and excessive side effects. They believe men will be better off avoiding unnecessary treatment even if it leads to an increased risk of progression and death from prostate cancer. Without screening men have the choice of treatment when symptoms appear or avoiding all surgery and radiation treatment. No primary treatment has been common in some Scandinavian countries and is often recommended by U.S. doctors for men with short life expectancies. Excessive risk of suffering and death from prostate cancer are the disadvantages of treatment avoidance strategies. No screening seems like an overreaction to the excessive treatment and side effects of cancer dominated strategies. Many men don't like choosing between these strategies and are looking for a better way to deal with prostate cancer.

We recommend the Best Life Strategy. It offers excellent cure rates coupled with avoidance of unnecessary side effects. Optimal screening provides early warning of progression and allows treatment of only progressing cancer while the cure rate is still high. The Best Life Strategy is compelling and practical. In one approach, men could shift the focus of their screening to progressing cancer only rather than all cancer that is usually dormant. We expect many doctors to recommend this strategy once they understand its power and practical advantages. Later in this disclosure we will evaluate all the strategies for a typical man.

### Strategy System

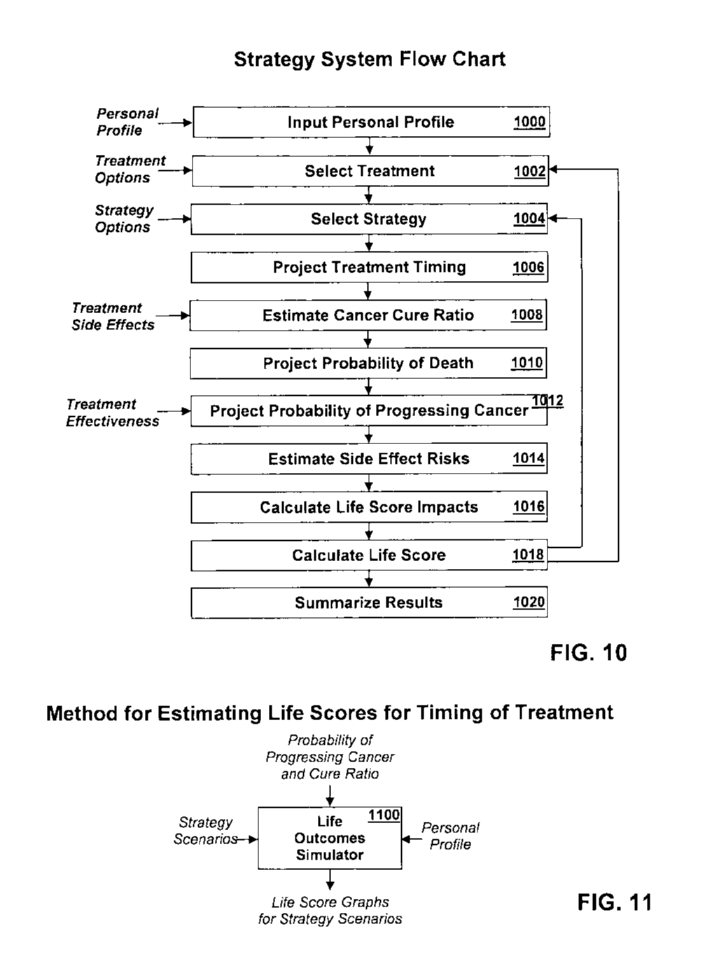

The methods and systems are introduced briefly below along with a high level flow chart on FIG. 10. Entering personal Profile information starts the analysis process at step 1000. Treatment is selected for analysis at step 1002 by the user or many treatments are analyzed in iterative fashion by the system. Opposing groups of doctors offer cancer dominated strategies that lead to unnecessary treatment and side effects or treatment avoidance strategies that lead to excessive risk of progression and death. The Best Life Strategy is optimal. The system analyzes a range of strategies in iterative fashion that are selected at step 1004. The annual probability of treatment for each future year is projected at step 1006 based on the probability of the detection of progressing cancer based on the man's risk profile and the amount of early or late warning implicit in the strategy. The Cancer Cure Ratio is estimated for treatment each year at step 1008 based on the amount of early or late warning implicit in the strategy. The Cure Ratio is used to project the probability of recurrence after treatment over time and subsequent progression at step 1010. The probability of death from prostate cancer is projected from the risk of subsequent progression for each year of potential treatment and then cumulated for an overall probability projection at step 1012. The risk of death from other causes is considered in estimating the increase in the overall risk of death for each future year. For each year of treatment the probability of treatment in that year is used to weight the subsequent risk of side effects. The risks for each year of treatment are cumulated to estimate an overall risk of side effects for each future year at step 1014. At step 1016 changes in Life Score are calculated for the increased risk of death by year and for the risk of side effects using the Emotional Weights entered by the user the his Personal Profile. The man's overall Life Score is reduced by the Life Score Impacts of increased risks of death and side effects at step 1018. Results are summarized for each strategy at step 1020.

### Life Score Calculation

Our life outcomes simulator, shown on FIG. 11, is used to calculate Life Score Impacts in module 1016 and Life Scores in module 1018 on FIG. 10 for a range of strategy scenarios. The probability of progressing cancer from a previous module is an example input. The user may supply information on his personal Profile. The system may supply a standard range of strategy scenarios.

### Strategy System Output

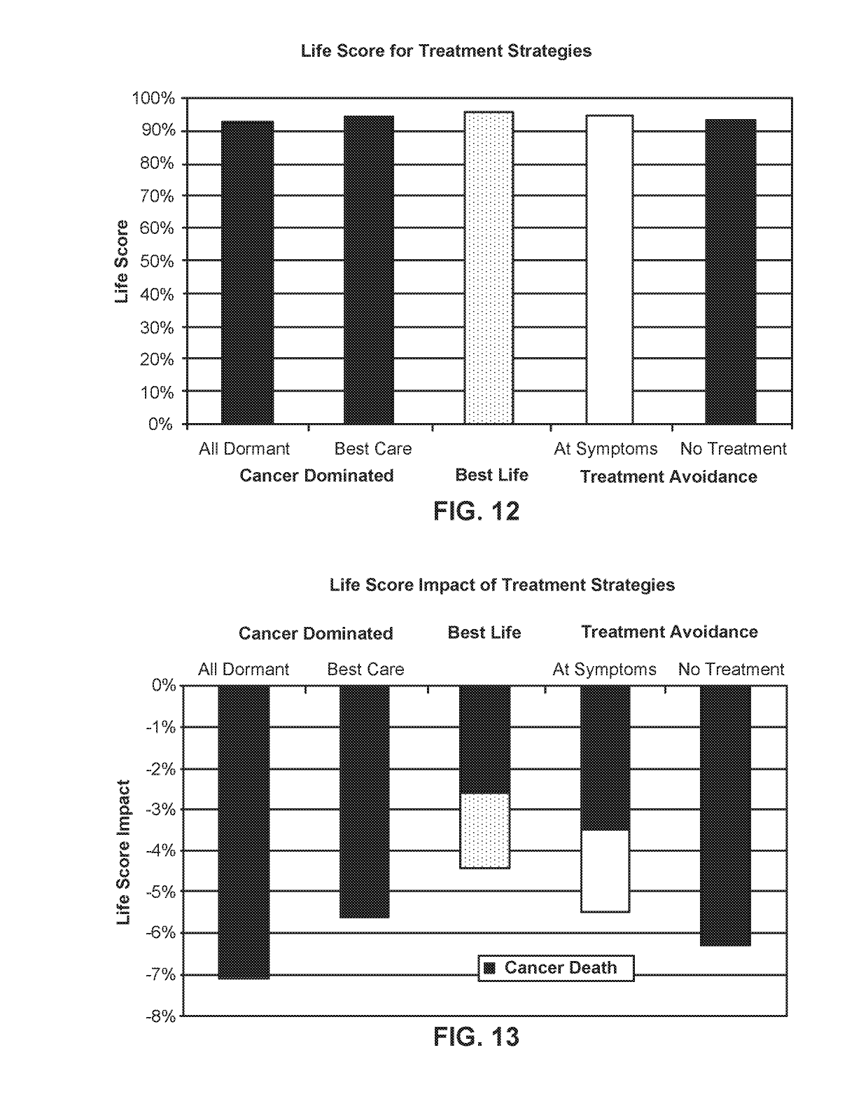

The Life Score Graph on FIG. 12 shows the Life Scores for a typical man for each treatment strategy. A value of 100% represents the Life Score in the absence of prostate cancer and serves as a point of reference. The graph on FIG. 12 shows the strategy that maximizes Life Score based on information entered in the Profile. Life Score summarizes well-being and lifespan. Treatments with the same Life Score may actually represent a tradeoff between different well-beings and life spans. Differences in Life Score may be interpreted in the context of a man's total life. For example, if he expects to live thirty more years, a 3% difference in Life Score would be equivalent to almost 1 year of his life.

Life Score Impact is the reduction in Life Score from side effects and death from prostate cancer. It measures the drop from 100% on the Life Score graph on FIG. 12. The graph on FIG. 13 shows the Life Score Impact of the treatment strategies. The total impact bars are split into a black section that shows the Life Score impact of death from prostate cancer and the lighter (colored coded) section that shows the Life Score impact of side effects from treatment. The bar with the smallest impact represents the treatment strategy with the highest Life Score.

### IV. Treatment Timing System

The Treatment Timing System helps men and their doctors choose time for treatment of prostate cancer that offers the highest Life Score. The Treatment Timing System builds on the results of the Dynamic Screening System.

### Competing Life Score Impacts

The timing of treatment for prostate cancer is a balancing act. Early treatment increases the chance of cure but may increase the risk of unnecessary treatment and side effects.

### Timing System

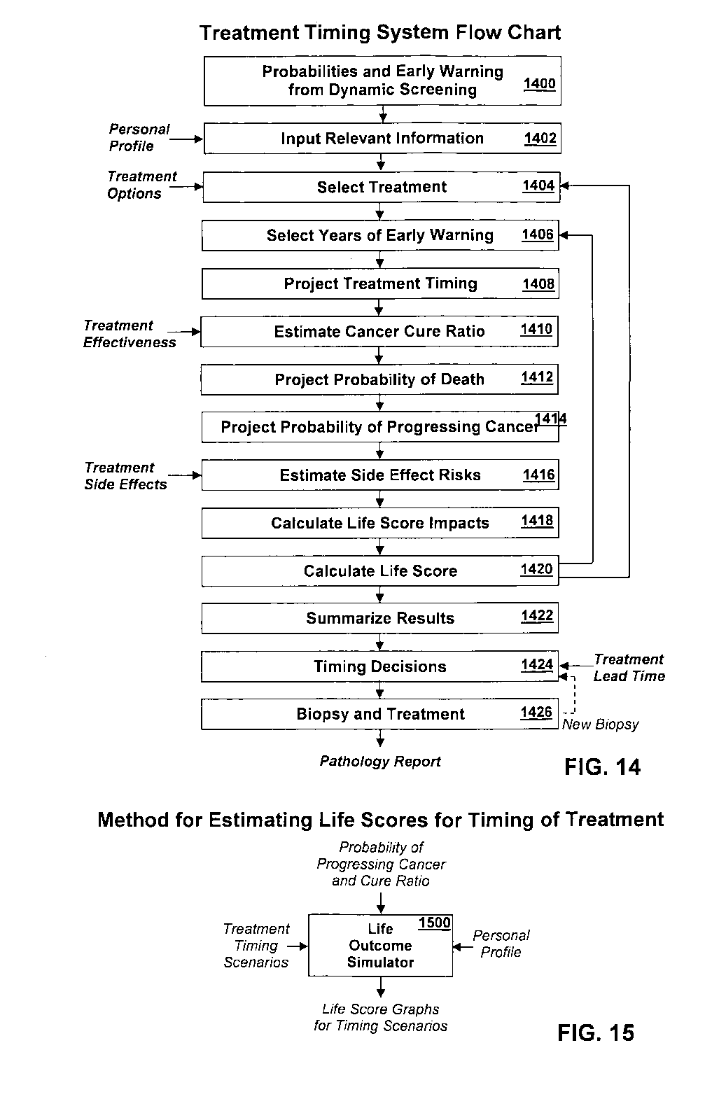

The methods and systems are introduced briefly below along with a high level flow chart on FIG. 14. The Probabilities and Early Warning results from Dynamic Screening are an input to the Treatment Timing System at step 1400. Other relevant information including personal Profile information is entered at step 1402. Treatment is selected for analysis at step 1404 by the user or treatments are analyzed in iterative fashion by the system. The system analyzes a range of years of early and late warning in iterative fashion from step 1406. The annual probability of treatment for each future year is projected based on the current probability of progressing cancer and years of early warning from the Dynamic Screening System in step 1408. The Cancer Cure Ratio is estimated for treatment each year at step 1410 based on the amount of early or late warning. The Cure Ratio is used to project the probability of recurrence after treatment over time and subsequent progression at step 1414. The probability of death from prostate cancer is projected from the risk of subsequent progression for each year of potential treatment and then cumulated for an overall probability projection at step 1412. The risk of death from other causes is considered in estimating the increase in the overall risk of death for each future year. For each year of treatment the probability of treatment in that year is used to weight the subsequent risk of side effects. The risks for each year of treatment are cumulated to estimate an overall risk of side effects for each future year at step 1416. At step 1418 changes in Life Score are calculated for the increased risk of death by year and for the risk of side effects using the Emotional Weights entered by the user in his Personal Profile. The man's overall Life Score is reduced by the Life Score Impacts of increased risks of death and side effects at step 1420. Results are summarized for each strategy at step 1422. A man, his doctor and his family can use Life Score simulations to help them choose the best timing for biopsy and treatment of progressing cancer (1424). For a biopsy, a doctor uses a device to inject thin hollow needles into the prostate to extract tissue. A pathologist exams the tissue and provides a diagnosis of prostate cancer if it exists. Primary treatment is intended to cure prostate cancer. It includes surgery to remove the prostate and various types of radiation to kill the cancer. A pathology report after surgery can provide useful information about the progress of cancer.

### Life Score Calculation

A life outcomes simulator, shown on FIG. 15, is used to calculate Life Score Impacts in module 1418 and Life Scores in module 1420 on FIG. 14 for a range of treatment timing scenarios. The probability of progressing cancer from a previous module is an example input. The user may supply information on his personal Profile. The system may supply a standard range of treatment timing scenarios.

### Maximum Life Score

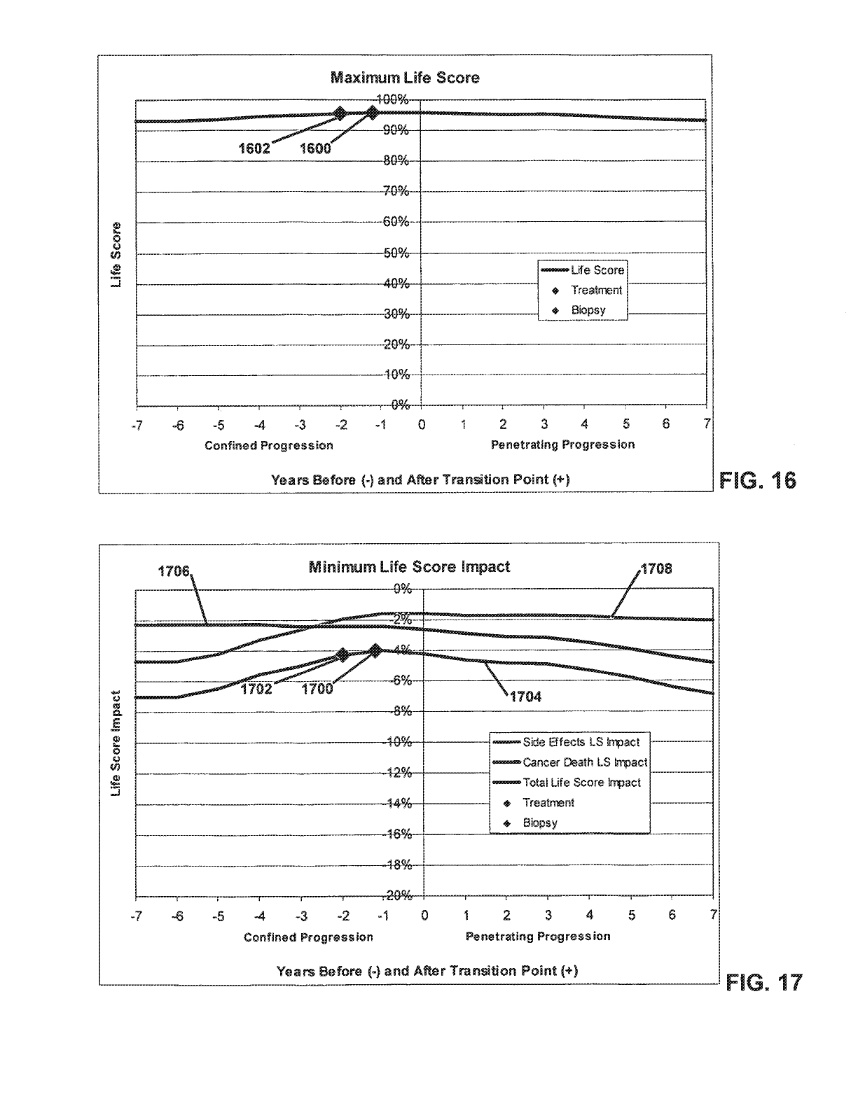

Life Score is a measure of well-being and length of life, based on the information entered in the Profile. The Life Score graph on FIG. 16 shows how Life Score varies for a range of treatment timing. A value of 100% represents Life Score in the absence of prostate cancer and serves as a point of reference. The Life Score curve is relatively flat because timing of prostate cancer treatment causes relatively small changes in well-being and length of life. Timing is measured in years before and after the Transition Point of progressing cancer (year 0). Before the Transition Point the Cure Ratio declines relatively slowly. After the Transition Point the Cure Ratio drops more steeply as the risk increases that cancer has spread outside of the prostate.

The color of the line and treatment diamond (1600) on the graph on FIG. 16 may depend on the primary treatment selected in the Profile purple for surgery, red for dual radiation, light orange for seed radiation and dark red for external radiation. The treatment diamond (1600) on each graph shows the treatment timing that maximizes Life Score and minimizes Life Score impact. For Life Scores that are different, one way to interpret the difference is in the context of a total life. For example, if someone expects to live thirty more years, a 3% difference in Life Score would be equivalent to almost 1 year of life.

The green diamond (1602) on each graph shows a rough estimate of biopsy timing that corresponds with the treatment timing that maximizes Life Score. A first biopsy should occur roughly six months to a year before the optimal time for treatment, so the biopsy timing diamond will show up on the graphs approximately six months to a year or more before the treatment timing diamond. The actual size of the biopsy lead time depends on a variety of factors.

### Minimum Life Score Impact

Life Score Impact is the reduction in Life Score by side effects and death from prostate cancer. It measures the drop from 100% on the Life Score graph of the previous section and allows us to magnify the changes shown. The graph on FIG. 17 shows the Life Score Impact for the range of treatment timing.

The bottom colored curve (1704) shows the total Life Score Impact for the treatment you chose. It is the sum of reduction in Life Score from side effects and death from prostate cancer. The curve is more pronounced than on the previous graph because the scale has been expanded. It does not span the full range of possible impacts from 0% to 100%.

The treatment diamond (1700) on each graph shows the treatment timing that maximizes Life Score and minimizes Life Score impact. The green diamond (1702) on each graph shows a rough estimate of biopsy timing that corresponds with the treatment timing that maximizes Life Score.

The gray curve (1708) shows the Life Score Impact of all side effects. The impact is greatest on the left when the risk of unnecessary treatment is greatest.

The black curve (1706) shows the Life Score Impact of death from prostate cancer. The impact is greatest on the right when late treatment leads to a decrease in cure rate and an increased risk of cancer death.

### V. Dynamic Screening System

The Dynamic Screening System helps men and their doctors screen for progressing cancer, long-term conditions and short-term conditions. It provides early warning of progressing cancer while reducing the probability of unnecessary treatment and side effects. The results are useful inputs to the Optimal Treatment Timing System. The prostate is the organ of the body used in the examples. Conditions used as examples are progressing prostate cancer, prostate volume growth caused by Benign Prostatic Hyperplasia (BPH) and infections of the prostate. Both PSA and Free PSA tests can be used for screening. Other tests may supplement them or replace them.

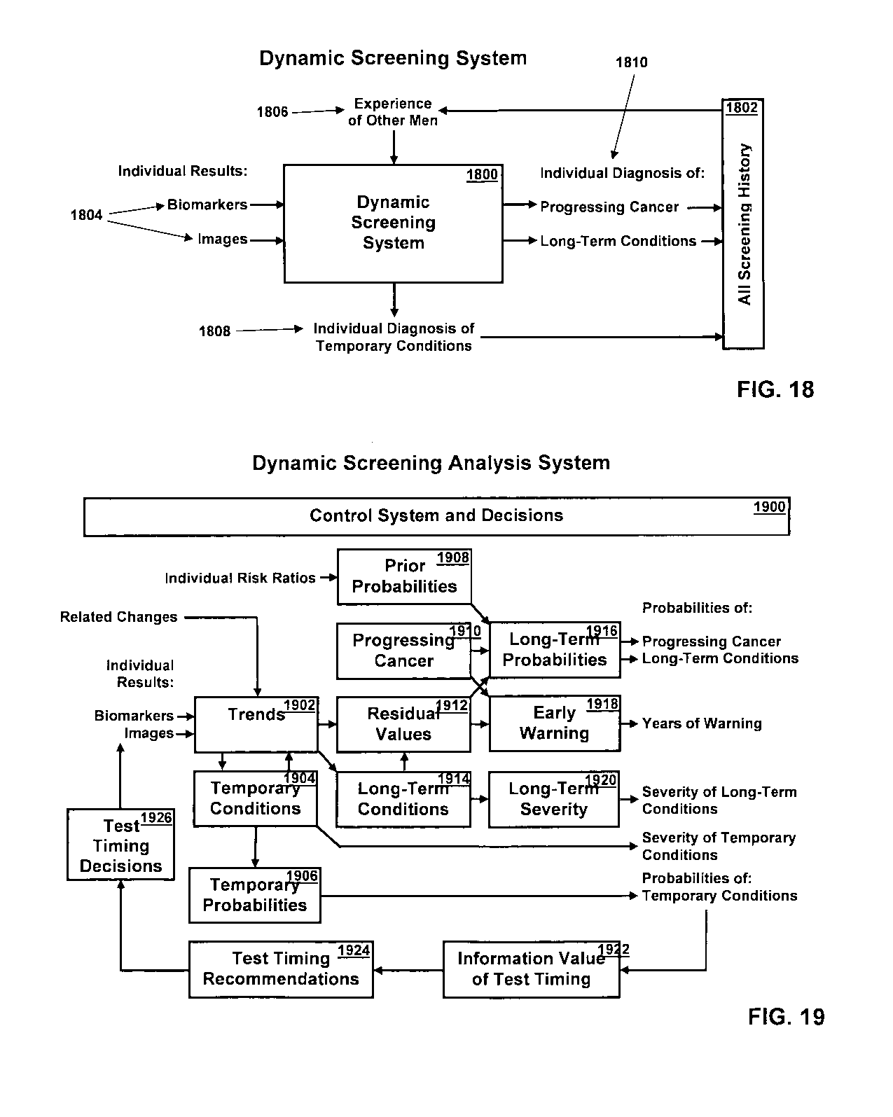

The flow chart on FIG. 18 provides a high level overview of the Dynamic Screening System. For one person, biomarker and image results are input on the left (1804). For the prostate, they are PSA and Free PSA test results and ultrasound measurements of prostate volume. The experience of other men is input from the top (1806). A diagnosis of temporary conditions comes out the bottom (1808). For the prostate, an infection is the most common and serious temporary condition. Diagnoses of progressing cancer and long-term conditions (volume growth due to BPH for the prostate) are output on the right (1810). All output becomes part of all screening history (1802) and is fed back as the experience of other men to increase the power of Dynamic Screening (1806).

The flow chart on FIG. 19 shows some of the modules of the Dynamic Screening System.

A man or his doctor registers him as a new user and completes the Profile for him. The man analyzes his strategy alternatives using the Prostate Strategy System and chooses the Best Life Strategy.

Using the Dynamic Screening System, the man follows suggestions about the type and timing of primary and secondary screening tests. Typically the system will recommend a baseline prostate volume study and annual PSA and Free PSA tests. Free PSA tests are currently recommended; however, other tests may be recommended in the future in conjunction with Free PSA or to substitute for it. Tests results will be entered into the system for analysis and guidance. Steadily increasing PSA due to prostate enlargement from BPH, if rapid enough, will lead the system to suggest periodic prostate volume measurements to define the rate of growth. Tests results will be entered into the system for analysis and guidance.

The Dynamic Screening System will recognize the false alarms caused by infection and other temporary conditions, provide calming perspective, suggest new PSA and Free PSA tests after the infection or condition has passed, and analyze the results of new tests.

The Dynamic Screening System will recognize early warning of possible cancer progression and suggest additional confirmation tests. Confirmation tests may include other components of PSA such as Pro PSA and any other useful new markers developed in the future. In addition, a new prostate volume study may be suggested, perhaps using more expensive technology if rapid prostate enlargement is a factor. A second round of confirmation tests will be suggested—perhaps six months after the first. Additional confirmation tests will be suggested until progression has been confirmed or rejected.

The Dynamic Screening System will confirm a high probability of progressing cancer when its calculation shows the probability is high enough to warrant consideration of biopsy and treatment

The Optimal Treatment Timing System will calculate the optimal schedule for biopsy and treatment based on ongoing screening tests and the information entered in the Profile. The man and his advisors will use the results to schedule a first biopsy and subsequent treatment.

In our feedback learning process, the man or his doctor will provide follow up information for the system to analyze and incorporate for use by other men.

### Control System and Decisions

The Control System and Decisions module (1900) and related modules (1922, 1924, 1926) on FIG. 19 help control the processing in the system and help men and their doctors makes decisions about testing.

### Control Systems and Decisions Module

The Control System and Decisions module (1900) helps guide the user and control the system. Annual blood tests should start at age 50 and perhaps earlier. Additional blood tests may be advised in the cases of temporary infection and increased probability of progressing cancer. A baseline prostate volume study should be performed in conjunction with the first blood tests. Additional tests may be needed if the probability of progression increases and/or prostate volume appears to be increasing rapidly. Currently both PSA and Free PSA tests are advised for early warning. Other types of tests are recommended to confirm or reject early warning. Ultrasound measurement of prostate volume and perhaps other secondary tests are recommended to help predict the consequences of prostate enlargement from BPH and other ongoing conditions that affect primary test results.

### Screening Decision Examples

There can be four decision points embedded in the ongoing screening process: Reject False Alarms. Escalate at Early Warning, Confirm Early Warning, and Rely on Backstop Warning.

Infections and other transitory events can cause jumps in PSA and raise false fears of prostate cancer for men. These PSA scares are common and troubling. One or more of our approaches can be configured to clearly identify these false alarms for what they are and avoid unnecessary fear and concern. They also allow rejection of the tests that caused the false alarms and their replacement by new tests after the infection has been eliminated or transitory event has passed.

Significant drops in the ratios of Free PSA to Total PSA can provide early warning of progressing cancer. Average Free PSA % drops very slowly, and Free PSA velocity % drops somewhat faster. Residual Free PSA velocity % is based on predictions for no cancer progression and provides much clearer early warning. We suggest adding additional blood tests as soon as early warning is noted. Over time the additional tests can confirm progression or reject it as a false warning.

Other markers for prostate cancer are under development. Some are being tested and may soon be available commercially, if they are not already. For example, Pro PSA has shown promise in studies reported in medical journals and others are in the pipeline. Our system will suggest additional tests be considered to confirm, or refute, early warning. The initial drop in Free PSA % ratios may be an accident, but a continued drop accompanied by shifts in ratios for other tests for can confirm a high probability of progressing cancer. Recall the adage that once may be an accident, twice a coincidence but three times is enemy action—with prostate cancer as the enemy in this case. Residual Free PSA velocity % may drop and give early warning four years before (−4) the cancer Transition Point. Additional confirming blood tests are administered soon after followed by more tests in each subsequent year Residual velocity % s for XPSA and YPSA can be calculated using the second test in year−3. A drop in all residual velocity % s will help confirm progressing cancer. The ratios for actual tests may start higher or lower than for Free PSA and may move more or less for progressing cancer—or even in the opposite direction. There can be a comparison of how the ratios actually move compared to the expected movement for both progressing cancer and its absence.

Confirmation tests can increase the probability of progressing cancer enough to suggest a biopsy followed by optimal timing for treatment or only enough to intensify the screening process. A high but not sufficiently high probability can lead to suggestions for more frequent blood testing and additional prostate volume studies, perhaps using more accurate MRI imaging, in order to more accurately estimate the probability that cancer is progressing.

For most men, early warning and confirmation tests will allow appropriately early treatment of progressing cancer, but there is a small chance that they will not provide adequate confirmation. Fortunately, residual PSA velocity provides extremely strong backstop warning of progressing cancer and makes it very hard to miss optimal timing by very much. It is hard to miss the steep increase in residual PSA velocity that typically occurs several years before the Transition Point when the Cure Ratio begins to drop steeply.

### Information Value of Test Timing Module

The Information Value of Test Timing module (1922) on FIG. 19 assesses the value of additional tests based on the existing output of the system and possible simulations of the impact of additional tests. For example, it may create a dummy pair of PSA and Free PSA tests, run the system with and without them included and observe the reduction in trend uncertainty from the additional test pair.

### Test Timing Recommendations Module

Test decision recommendations about the type and timing of primary and secondary tests are created in the Test Timing Recommendations module (1924) on FIG. 19. Examples of recommendations are presented below.

The system may suggest base tests starting at age 50 or earlier based on risk factors and personal preference. They might consist of: Primary Tests—Two annual blood tests: Total PSA and Free PSA; and Secondary Tests—One baseline prostate volume study.

The system may suggest next tests based on evaluation of the most recent tests: Base tests, Prostate enlargement tests, Retests after false alarms, and Confirmation tests after early warning.

The system may suggest periodic prostate volume studies for men with rapidly growing prostates due to BPH. For some men with extremely high growth rates the system will suggest more accurate volume studies using MRI or other high accuracy imaging.

In the Test Validity Test step the system may recognize the false alarms raised by PSA scares from infections and other temporary conditions. New PSA tests will be suggested after the condition has passed.

The Probability Estimate step may provide early warning of the possibility of progressing cancer. Additional primary tests will be suggested when early warning of progressing cancer is recognized. Additional and perhaps more accurate volume studies may be suggested for men with prostates growing from BPH.

### Test Timing Decisions Module

The user can explore the implications of the number and timing of additional tests in the Test Timing Decisions module (1926) on FIG. 19. Among other capabilities, the user can submit hypothetical results to discover the relative value of different combinations of tests and timing.

### Trends and Temporary Conditions

Three related modules of the Dynamic Screening System (FIG. 19) are described in this section: the Trends module (1902), the Temporary Conditions Module (1903) and the Bayesian Probabilities module for temporary conditions (1904).

### Trends Module

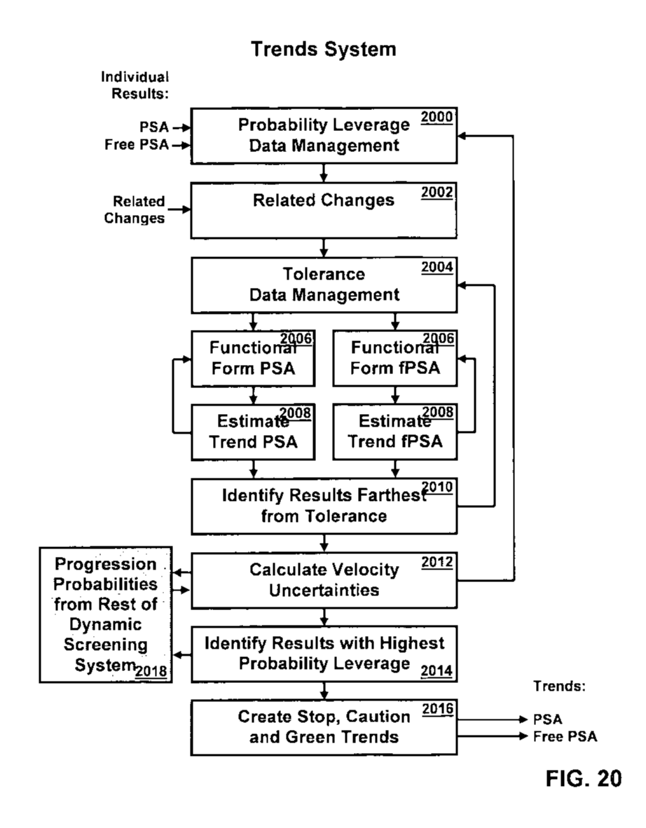

The Trends module (1902) on FIG. 19 estimates trends in PSA and Free PSA after excluding results that are outside of a reasonable tolerance range. Details of the Trends module are shown on FIG. 20 and described in the following sections.

### Probability Leverage Data Management Module

The Probability Leverage Data Management module (2000) starts the trends process and controls its outer loop of iterations. Inputs may include: PSA and Free PSA dates and test results as entered by the user and feedback of the pair of highest leverage test results to remove from next the iteration. Outputs may include: PSA and Free PSA dates and test results to be used for each trend—the Red Stop trend, the Yellow Caution trend and the Green trend. These trends offer different amounts of sensitivity to anomalous tests and early warning. The module controls the outer iterative loop to find the pair of test results that creates the largest increase in the probability of progressing cancer. The first pair removed drops the trend from the red stop trend to the yellow caution trend. The second pair removed drops the trend from the yellow caution trend to the green trend.

### Related Changes Module

The Related Changes module (2002) may adjust test results for related changes with known impacts. Some treatments and changes in medication and life style can affect the level of PSA and the results of other screening tests. For example, treatments to reduce the size of the prostate, like a TURP, can cause a sharp drop in the level of PSA and other markers. If entered into a man's Profile, related changes can be used to adjust the test results, trends and predictions of PSA and other variables after the change.

Some treatments for prostate cancer conditions can significantly alter the production of PSA and other screening variables. The results of the change can be predicted based on the experience of other men. Some medications can alter the production of PSA and other screening variables. The results of the change can be predicted based on the experience of other men. Some changes in lifestyle can alter the production of PSA and other screening variables. The results of the change can be predicted based on the experience of other men for changes in: Diet, Exercise, Supplements, Recreational Drugs and also the brand and method the tests.

### Tolerance Data Management Module

The Tolerance Data Management module (2004) controls the inner loop that excludes test results until all included test results are within the tolerance region of the trends. Inputs may include: PSA and Free PSA dates and test results to be used for each trend: the Red Stop trend, the Yellow Caution trend and the Green trend; and Feedback about the test Pair farthest from the tolerance test region to remove from next iteration. Output may included: PSA and Free PSA dates and test results to be used for each tolerance test trend.

The module controls the inner loop of trend calculations to find trends that fit the data with all test results within the tolerance region. Pairs of tests farthest from tolerance by ratio are removed during each iteration until all remaining test results are within the tolerance region.

### Functional Form for PSA and Free PSA Modules

The Functional Form PSA and Functional Form fPSA modules (2008) may change the functional forms used to fit the trends based on the number and duration of test results, the characteristics of the trends and other factors that may be relevant. Inputs may include: PSA and Free PSA dates and test results to be used for each tolerance test trend; and feedback about PSA Velocity and Free PSA Velocity from the estimated trends. Output may include: Functional forms used to estimate PSA and Free PSA trends and constraints on function parameters.

The module may select functional forms used to estimate trends for PSA and Free PSA and constrain the parameters used. Example selection rules might include:
One data point
   No trend is estimated.
Two data points
   Linear trend is estimated (two parameters)

PSA(t)=a+b*t

fPSA(t)=a+b*t
Three or more data points through five years
   Power-law trend is estimated (three parameters)

PSA(t)a+b(t−to)̂c

fPSA(t)=a+b*(t−to)̂c
Power-law parameter (c) is constrained to limit curvature
   c increases with number of test results
   c increases with duration of test period up to five years
   c depends on feedback of velocity from estimated trend
Six or more data points and five to seven years
   Power-law trend from above is used for the most recent five years.
   Linear trend is used from the start of tests to five years before the last test.
      Linear trend equals power-law trend at year five.

PSA(t)=a+b*t

fPSA(t)a+b*t
Slope (b) is estimated to fit data (one new parameter)
Level (a) is adjusted so linear trend equals power-law at year five.
Eight or more data points and more than seven years
   Power-law trend from above is used for the most recent five years.
   Linear trend is used from half way between the start of tests and five years before the last test to five years before the last test, as above.
   Linear trend is used from the start of tests to the halfway point.
      Linear trend equals next linear trend at halfway point.

PSA(t)=a+b*t

fPSA(t)=a+b*t
Slope (b) is estimated to fit data (one new parameter)
Level (a) is adjusted so first linear trend equals second linear trend at halfway point.
   The rationale for some functional forms is presented as an example. A line is the only trend we can fit when we have only two data points. Dynamic Screening may fit a curved trend to curved data, including accelerating PSA. The power-law function has advantages over the quadratic and other three parameter curved functions because it offers the most power for progressing cancer with little significant compromise for no progression conditions. For these reasons, the power-law function seems the dominant choice because it is the functional form of progressing cancer, our focus. Moreover, it has an intuitive, one parameter way of constraining curvature.
   The power-law function can be constrained to a linear function by setting (c)=1.0. Curvature can be limited by constraining the range of values allowed for (c),
Power-law trend is estimated (three parameters)

PSA(t)=a+b*(t−to)̂c

fPSA(t)=a+b*(t−to)̂c
Power-law parameter (c) is constrained to limit curvature
   c increases with number of test results
   c increases with duration of test period up to five years
   c depends on feedback of velocity from estimated trend
      Decisions about limitation on curvature are a balancing act. Tight limits prevent a few data points leading to an estimated trend with far more curvature than is likely but may cause some underestimation of unusually large curvature.

### Estimate Trends for PSA and Free PSA Modules

The Estimate Trends for PSA and Free PSA modules (2008) may use a variety of methods to estimate trends for trends included in the iteration controlled by the Tolerance Data Management module (2004). Inputs may include: PSA and Free PSA dates and test results to be used for each tolerance test trend; and the functional forms used to estimate PSA and Free PSA trends. Output may include: PSA and Free PSA trends; and Feedback about PSA Velocity and Free PSA Velocity from estimated trends. Least squares methods may be used to estimate the parameters for the chosen functional form that best fits the test results. If the unconstrained estimate of (c) for curvature is within the constraints then it is used. If the unconstrained estimate of (c) is beyond a constraint then the constraint is used for (c) and the trend is re-estimated.

The trend equations may be used to define trend values at each test date, including projections to the most recent test dates.

Identify Results Farthest from Tolerance Module

The Identify Results Farthest from Tolerance module (2010) determines which test pairs are candidates for exclusion from trend estimation on the next iteration controlled by the Tolerance Data Management module (2004). Inputs may include: PSA and Free PSA trends, and PSA and Free PSA dates and test results to be used for each tolerance test trend. Outputs may include: Feedback about the Pair of PSA and Free PSA tests farthest from tolerance (for removal); and PSA and Free PSA trends that have all test results within tolerance and the PSA and Free PSA dates and test results within tolerance of trends.

The tolerance region for each test date may be a two dimensional region around the trends defined by two variables like PSA and Free PSA or one variable like PSA and the ratio of one variable to the other like Free PSA divided by PSA. Most of the test results within the tolerance region may be explained by random variation. Most of the test results outside of the tolerance region may be explained by short-term conditions. The shape of the tolerance region may approach a rectangle for highly correlated dimensions, may approach an oval for relatively uncorrelated dimensions or be something in between. The shape of tolerance region may be adjusted to produce unbiased trends that are not distorted by temporary conditions. For example, infections are the most prevalent temporary condition and cause PSA to increase and the Free PSA % to decrease. Some of the area of the tolerance region in that direction may be reduced in order to reduce the bias caused by infections.

For each tolerance iteration, pairs of PSA and Free PSA tests are compared to the estimated trends. Beyond-tolerance pairs that are farthest by ratio from the trend at the test date are identified for removal from the next tolerance iteration through the feedback process. The tolerance iteration process stops the first time all pairs are within tolerance. The trends and remaining pairs are output to the next process step.

An oval or ellipse shape may be appropriate for relatively uncorrelated variables such as PSA and Free PSA %. An elliptical tolerance region may be calculated in the following way. With FreePSA on the Y axis and PSA on the X axis of a coordinate plane, the upper half of an ellipse is defined by

$$y = f + \sqrt{{b}^{2} ( 1 - \frac{{( x - p )}^{2}}{{a}^{2}} )}$$

where f=FreePSAtrend, p=PSAtrend, b=(some tolerance value*FreePSAtrend), and a=(some tolerance value*PSAtrend). If the FreePSA point is less than the FreePSAtrend value, FreePSA′ is defined as (FreePSAtrend+(FreePSAtrend−FreePSA)), otherwise FreePSA′ is FreePSA. If the point (PSA, FreePSA′) is above the ellipse then both tests are excluded, otherwise both tests are included.

### Calculate Velocity Uncertainties Module

The Calculate Velocity Uncertainties module (2012) may calculate velocity uncertainties for each trend for use by the Bayesian calculation of the probability of progressing cancer. Inputs may include: PSA and Free PSA trends from the removal of each possible high leverage pair; and PSA and Free PSA dates and test results with each possible high leverage pair. Outputs may include: Feedback: PSA and Free PSA velocity uncertainties to the rest of the system; PSA and Free PSA trends with the highest leverage pair removed; and PSA and Free PSA dates and test results with the highest leverage pair removed.

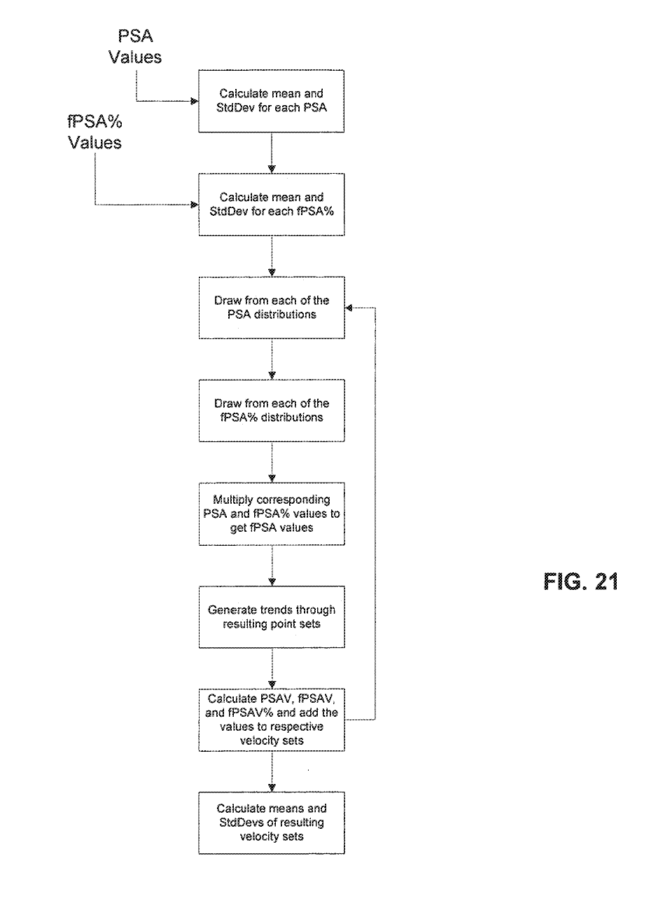

A variety of methods can be used to calculate uncertainties in the velocities, like PSA Velocity, and velocity ratios, like Free PSA Velocity %, which is the ratio of Free PSA Velocity to PSA Velocity. Monte Carlo methods may be used to calculate the velocity distributions for each variable around the trends, as shown on FIG. 21. Probability distributions for the variables may come from studies of other men or the experience of the man under consideration. In the case of Free PSA, variation in Free PSA is correlated with variation in Free PSA so this relationship is considered by the method used to estimate the distribution of Free PSA % and Free PSA in light of the randomly drawn corresponding PSA result.

Identify Results with Highest Probability Leverage Module

The Identify Results with Highest Probability Leverage module (2014) may determine which test pairs seem to be most anomalous and are the best candidates for elimination to create the yellow Caution trend and the Green trend. Test pairs may be tested for impact on the probability of progressing cancer in real time using the rest of the system (2018) or using rules of thumb or reduced-form results based on off-line simulations using the rest of the system. Inputs may include: PSA and Free PSA trends that have all test results within tolerance; PSA and Free PSA dates and test results within tolerance of trends; and Probability of progressing cancer from the rest of the system. Outputs may include: Feedback about the Pair of highest leverage PSA and Free PSA tests results (for removal); PSA and Free PSA trends for Stop, Caution and Green trends; and PSA and Free PSA dates and test results for Stop, Caution and Green trends.

For the red Stop trends the first set of PSA and Free PSA trends may be passed through to the next step. The red Stop trend is most sensitive to anomalous test results but provides the earliest warning if cancer is progressing.

For the yellow Caution trends, the most likely high leverage pairs of PSA and Free PSA tests may be removed one at a time. Trends may be calculated and the results may be sent to the rest of the system for calculation of the probability of progressing cancer. The pair that causes the largest change in the probability of progressing cancer may be identified and removed. The trends estimated without that pair may be passed through to the next step as the yellow Caution trends. The removed pair may feed back to the probability leverage data management step for exclusion from the start of the Green trend iteration.

For the Green trends, the most likely high leverage pairs of PSA and Free PSA tests may be removed one at a time. Trends may be calculated and the results may be sent to the rest of the system for calculation of the probability of progressing cancer. The pair that causes the largest change in the probability of progressing cancer may be identified and removed. The trends estimated without that pair may be passed through to the next step as the Green trends.

In many cases significant probabilities of progression may not be calculated by the rest of the Dynamic Screening system. Velocities may be very low, or trend uncertainty may be high. In these cases, fall back methods may be used to estimate which pairs of results will tend to increase the probability the most. Pairs that increase PSA Velocity trends the most and decrease Free PSA Velocity % trends the most are likely to increase the probability the most. The relative impact of the two velocities may be calculated from the specific conditions or estimated from off line calculations for many men.

Progression Probabilities from Rest of the System

The trends and their uncertainties may be sent to the rest of the system (2018) for analysis and calculation of the probability of progressing cancer. This function may be performed in real time or as off-line simulations where the results are used as rules of thumb or reduced form models. Inputs may include: PSA and Free PSA trends from the removal of each possible high leverage pair; and PSA and Free PSA velocity uncertainties. Outputs may include: Probability of progressing cancer from the removal of each possible high leverage pair.

### Create Stop, Caution and Green Trends Module

The Create Stop, Caution and Green Trends module (2016) may collect, label and output the three trends for use by the rest of the system and for display, as well as pass them through to the next step. Inputs may include: PSA and Free PSA trends with the highest leverage pair removed; and PSA and Free PSA dates and test results with the highest leverage pair removed. Outputs may include: PSA and Free PSA trends for Stop, Caution and Green trends; and PSA and Free PSA dates and test results for Stop, Caution and Green trends.

### Temporary Conditions Module

The Temporary Conditions module (1904) shown on FIG. 19 may assess the possibility of temporary conditions and their severity. Inputs may include trends and pairs of test results. Outputs may include estimates of the severity of the temporary condition. Infection of the prostate is an example. Severity of prostate infections may be indicated by how much a PSA test result exceeds the value for that date predicted by the trend and by how much the corresponding test Free PSA % is below the value for that date predicted by the trends

### Temporary Probabilities Module

The Temporary Probabilities Module (1906) shown on FIG. 19 may estimate the probability of temporary conditions, like infection of the prostate. A variety of methods may be used to calculate the estimate of the probability, including Bayesian methods. In broad terms a prior probability of an infection is augmented by estimates of the probability of the observed test results, such as PSA and Free PSA %, given that the prostate is infected and given it is not infected.

### Long-Term Conditions Severity

The Long-Term Conditions Severity module (1920) on FIG. 19 estimates the severity of long-term conditions assuming cancer is not progressing. Growth in prostate volume is the long-term condition we are considering as an example. Prostate volume growth is caused by Benign Prostatic Hyperplasia and causes bothersome symptoms like frequent urination and difficulty urinating. The severity of the condition is measured by prostate volume measured in cubic centimeters and volume velocity measured by the increase in cubic centimeters per year.

### Volume Measurement

Prostate volume can be measured from images of the prostate. Ultrasound is the most common and cost effective imaging technique for measuring prostate volume, but MRI and other techniques can be used effectively. Multiple volume measurements over time are needed to fit a trend and estimate volume velocity. Every man should consider a baseline study done when he reaches age 50 or earlier for men with higher risk of cancer or a history of prostate enlargement. Volume studies can be as infrequent as every five years for men with no evidence of prostate enlargement and no indications of progressing cancer. More frequent studies are suggested for men with enlarging prostates due to BPH and with increasing probability of progressing cancer.

PSA trends can be used to estimate volume and volume velocity if no volume measurements are available and can be used in conjunction with one or more volume measurements to improve the estimates of volume velocity. Progressing prostate cancer increases PSA without increasing prostate volume significantly. Therefore, we explicitly assume that cancer is not progressing when we use PSA trends to estimate prostate volume trends. Progressing cancer is a competing hypothesis to explain increasing PSA.

### Multiple Volume Measurements

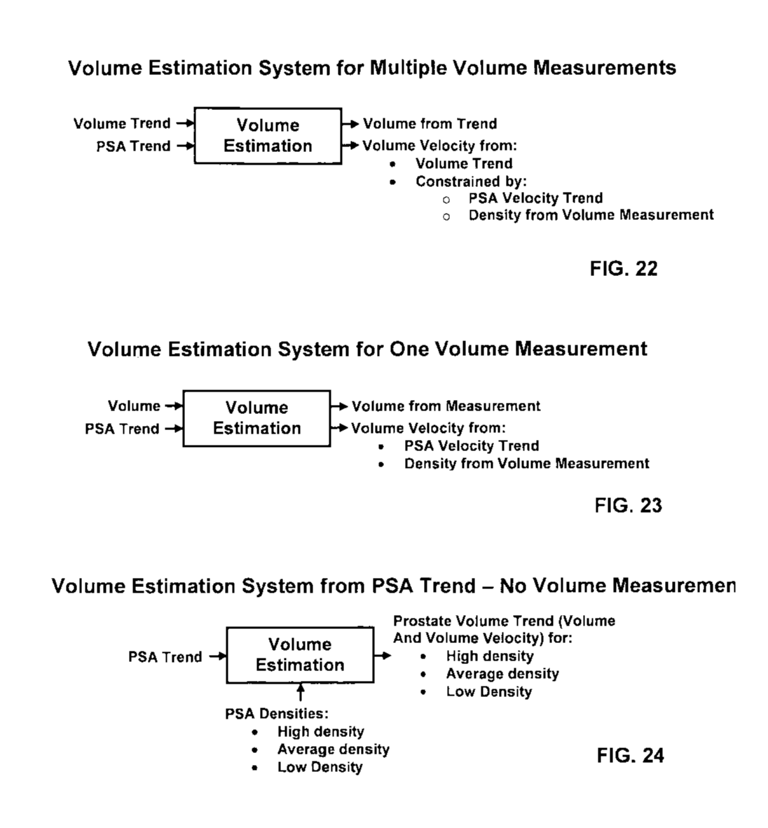

Multiple volume measurements are the best way to estimate a volume trend. The trend defines volume and volume velocity at each point between the first and last measurement and can be used to project them beyond the last measurement to the present. However, only two volume measurements over a short period of time can lead to unreasonably high or negative velocity estimates because of variation in volume measurements. Therefore, the Volume Estimation System uses the PSA Trend to constrain the range of reasonable volume velocities. The PSA trend values divided by volume trend values provides a good estimate of PSA density. PSA Velocities from the PSA trend divided by PSA density provide a good estimate of Volume Velocities, which can be used to constrain the overall estimate of Volume Velocities to reasonable values. FIG. 22 shows the Volume Estimation System for multiple volume measurements.

### One Volume Measurement

One volume measurement alone does not allow estimation of a volume trend, but it substantially improves it compared to estimates with no volume measurement. The Volume Estimation System uses the volume measurement and the PSA Trend to estimate the volume trend. The PSA trend value at the time of the volume measurement divided by the volume measurement provides a good estimate of PSA density. The PSA trend divided by PSA density provides a good estimate of the Volume trend, which can be used to project current Volume and Volume Velocity. FIG. 23 shows the Volume Estimation System for one volume measurement.

Volume Estimates from PSA Trend—No Volume Measurement

Many men have no volume measurement. The Volume Estimation System uses the PSA Trend and three typical PSA densities to estimate the volume trend. The densities are chosen from population data based on the man's age: average, high (upper 10^{th} percentile) and low (lower 10^{th} percentile). The PSA trend divided by one or more of the PSA densities provides one or more estimates of the Volume trend, which can be used to project current Volume and Volume Velocity. FIG. 24 shows the Volume Estimation System for no volume measurement.

### Long-Term Conditions

The Long-Term Conditions module (1914) on FIG. 19 predicts non-cancer primary test results based on past experience, recent secondary test results and the experience of other men. Prostate volume growth is the long-term condition of concern for the prostate. Volume growth is caused by Benign Prostatic Hyperplasia. PSA and Free PSA increase as the prostate grows.

Predicted PSA Velocity, Free PSA Velocity and Free PSA Velocity % provide a reference against which actual results can be compared and analyzed for progressing cancer. These predicted velocities are subtracted from trend velocities in order to calculate residual velocities. Free PSA is the preferred second screening test and is described below; however, the results of other screening tests can be predicted in the same way either as a substitute or complementary confirming test.

The prediction methods vary depending on the amount of data available. Prostate volume can be measured cost-effectively using ultrasound images and can be measured using MRI and other images. The method of predicting trends in PSA and Free PSA Velocities depends on the number of volume measurements available. Example methods are shown for three cases: multiple volume measurements, one volume measurement and no volume measurement. Multiple volume measurements are used to estimate a volume trend and calculate the corresponding volume velocity trend.

Predictions of velocities for no progressing cancer improve as length of the PSA and Free PSA testing history increases and the number of tests increases. Example methods are shown for two cases: long testing history and Short testing history. Not all combinations of volume measurements and testing history are shown, but they can be inferred from the examples shown.

Finally, the system estimates uncertainty in the predicted results measured by standard deviation in the predicted velocities. These standard deviations are inputs to the calculation of the probability of progressing cancer and the years of early warning for progressing cancer.

### Multiple Volume Measurements and Long Screening History

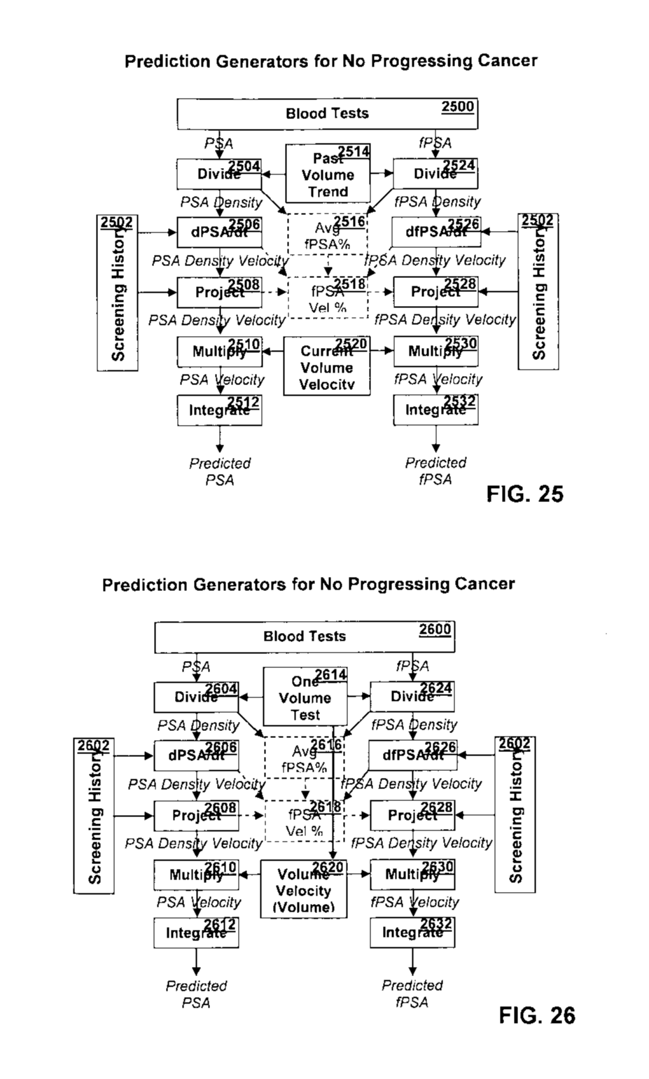

This method may apply when two or more volume measurements are available along with a long screening history, as shown on FIG. 25. A volume trend is estimated. Volume velocity is compared with population velocities for the same volumes and may be constrained for reasonableness.

In module (2500) PSA and fPSA blood test results are input to the method. The Volume trend is used to estimate a past volume (2514) in order to estimate past densities. Past PSA and fPSA trend results are divided (2504 and 2524) by past trend volume or volumes to calculate densities. Average values are calculated for fPSA % (2516), Free PSA divided by PSA, but they play a secondary role when a long screening history is available. Please see the short screening history example to learn about its stronger role. Velocities are calculated as annual changes—dPSA/dt (2506) and dfPSA/dt (2526). fPSA Vel % (2518) is calculated as Free PSA Velocity divided by PSA Velocity using a combination of projected PSA Density Velocity and projected fPSA Density Velocity. However, fPSA Vel % may play a secondary role in projecting fPSA Density Velocity when a long screening history is available. PSA Density Velocity (2508) is projected from past trends. fPSA Density Velocity (2528) is calculated using primarily the projection of Free PSA Density Velocity (2526) and secondarily the estimate of fPSA Vel % (2518). Current Volume Velocity (2520) is estimated from the Volume trend. A Volume Velocity trend is calculated from the Volume trend. PSA and Free PSA Velocities are calculated by multiplying (2510 and 2530) the current volume velocity (2520) times the projected density velocities (2508 and 2528). Predicted values for PSA and Free PSA with no progressing cancer are calculated by integrating (2512 and 2532) the PSA and Free PSA velocities and adding them to the PSA and Free PSA trend values at the start of the integration period.

### One Volume Measurement and Long Screening History

This method may apply when one volume measurement is available along with a long screening history, as shown on FIG. 26. A reduced form of this method may be used that predicts current PSA Velocity based on the volume measurement and PSA trend value at the time of the measurement and Free PSA velocity based on current predicted PSA Velocity and the Free PSA Velocity % trend.

In module (2600) PSA and fPSA blood test results are input to the method. The one Volume measurement (2614) is used to estimate past densities and to predict current volume velocity (2620). Past PSA and fPSA trend results are divided (2604 and 2624) by past volume to calculate past densities. Average values are calculated for fPSA % (2616), Free PSA divided by PSA, but they play a secondary role when a long screening history is available. Please see the short screening history example to learn about its stronger role. Velocities are calculated as annual changes—dPSA/dt (2606) and dfPSA/dt (2626). fPSA Vel % (2618) is calculated as Free PSA Velocity divided by PSA Velocity using a combination of projected PSA Density Velocity and projected fPSA Density Velocity. However, fPSA Vel % may play a secondary role in projecting fPSA Density Velocity when a long screening history is available. PSA Density Velocity (2608) is projected from past trends. fPSA Density Velocity (2628) is calculated using primarily the projection of Free PSA Density Velocity (2626) and secondarily the estimate of fPSA Vel % (2618). Current Volume Velocity (2620) is estimated from the one volume test (2614). PSA and Free PSA Velocities are calculated by multiplying (2610 and 2630) the current volume velocity (2620) times the projected density velocities (2608 and 2628). Predicted values for PSA and Free PSA with no progressing cancer are calculated by integrating (2612 and 2632) the PSA and Free PSA velocities and adding them to the PSA and Free PSA trend values at the start of the integration period.

### No Volume Measurement and Long Screening History

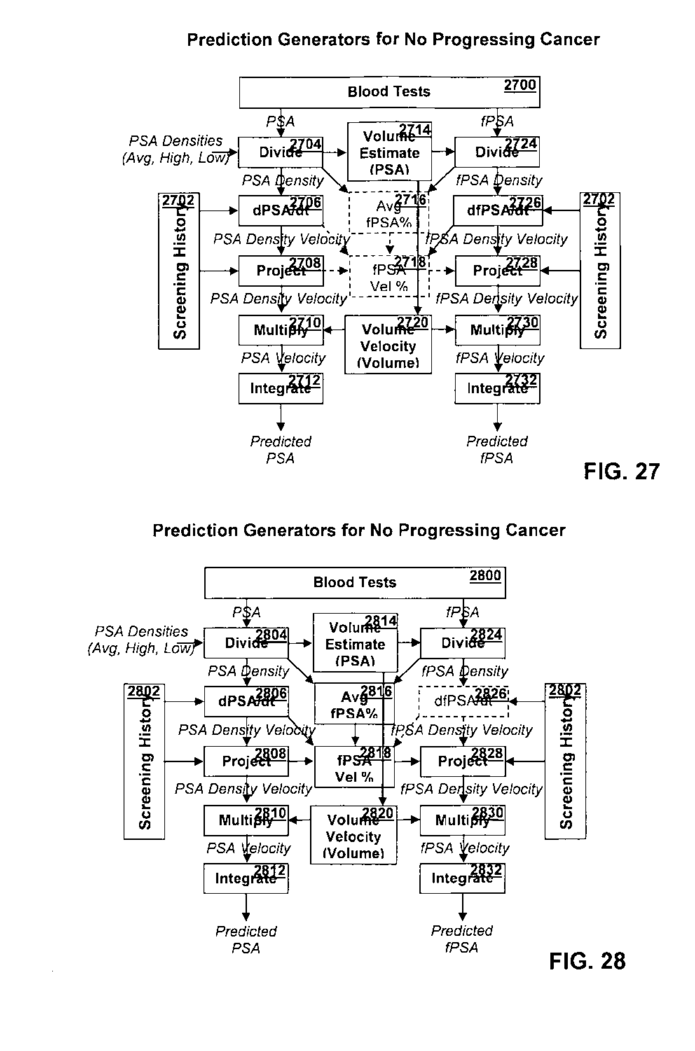

This method may apply when no volume measurement is available along with a long screening history, as shown on FIG. 27. A reduced form of this method may be used that predicts current PSA Velocity based on past PSA levels and Free PSA velocity based on current predicted PSA Velocity and the Free PSA Velocity % trend.

In module (2700) PSA and fPSA blood test results are input to the method. The PSA trend is divided (2704) by age specific population PSA densities to estimate prostate volumes (2714). This volume estimate is used to predict current volume velocity (2720). fPSA trend results are divided (2724) by volume estimates to calculate Free PSA densities. Average values are calculated for fPSA % (2716), Free PSA divided by PSA, but they play a secondary role when a long screening history is available. Please see the short screening history example to learn about its stronger role. Velocities are calculated as annual changes—dPSA/dt (2706) and dfPSA/dt (2726). fPSA Vel % (2718) is calculated as Free PSA Velocity divided by PSA Velocity using a combination of projected PSA Density Velocity based on population PSA densities and projected fPSA Density Velocity based on population PSA densities and fPSA Vel %. However, fPSA Vel % may play a secondary role in projecting fPSA Density Velocity when a long screening history is available. PSA Density Velocity (2708) is projected from past trends. fPSA Density Velocity (2728) is calculated using primarily the projection of Free PSA Density Velocity (2726) and secondarily the estimate of fPSA Vel % (2718). Current Volume Velocity (2720) is estimated from the volume estimate (2714). PSA and Free PSA Velocities are calculated by multiplying (2710 and 2730) the current volume velocity (2720) times the projected density velocities (2708 and 2728). Predicted values for PSA and Free PSA with no progressing cancer are calculated by integrating (2712 and 2732) the PSA and Free PSA velocities and adding them to the PSA and Free PSA trend values at the start of the integration period.

### No Volume Measurement and Short Screening History

This method may apply when no volume measurement is available along with a short screening history, as shown on FIG. 28. A reduced form of this method may be used that predicts current PSA Velocity based on past PSA levels and Free PSA velocity based on current predicted PSA Velocity and the Free PSA Velocity % trend or just the Free PSA % trend.

In module (2800) PSA and fPSA blood test results are input to the method. The PSA trend is divided (2804) by age specific population PSA densities to estimate prostate volumes (2814). This volume estimate is used to predict current volume velocity (2820). fPSA trend results are divided (2824) by volume estimates to calculate Free PSA densities. Average values are calculated for fPSA % (2816), Free PSA divided by PSA. This value is used as the estimate for fPSA Velocity % when no history of that variable is available for projection. Velocities are calculated as annual changes—dPSA/dt (2806) and dfPSA/dt (2826). dfPSA/dt may not be available. If available fPSA Vel % (2818) is calculated as Free PSA Velocity divided by PSA Velocity using a combination of projected PSA Density Velocity based on population PSA densities and projected fPSA Density Velocity based on population PSA densities and fPSA Vel %. However, fPSA Vel % may play a stronger role in projecting fPSA Density Velocity when a short screening history is available. PSA Density Velocity (2808) is projected from past trends. fPSA Density Velocity (2828) is calculated using primarily the projection of PSA Density Velocity (2808) and the estimate of Free PSA Velocity % (2818) and secondarily the estimate of fPSA Vel (2826). Current Volume Velocity (2820) is estimated from the volume estimate (2814). PSA and Free PSA Velocities are calculated by multiplying (2810 and 2830) the current volume velocity (2820) times the projected density velocities (2808 and 2828) Predicted values for PSA and Free PSA with no progressing cancer are calculated by integrating (2812 and 2832) the PSA and Free PSA velocities and adding them to the PSA and Free PSA trend values at the start of the integration period.

### Uncertainty in Predicted Values for No Progressing Cancer

The process for estimating the probability of long-term conditions like progressing cancer and volume growth depends on the total amount of uncertainty in the predicted PSA Velocity, Free PSA Velocity and Free PSA Velocity %. The system may estimate uncertainty in the predicted results using standard deviation in the predicted velocities.

Trend uncertainty and biologic uncertainty contribute to the total amount of uncertainty in PSA and Free PSA trends. Trend uncertainty is caused mostly by short-term biologic variation with some test measurement variation thrown in, module 1902 on FIG. 19. The other source of variation is long-term biologic uncertainty about volume growth. It reflects variation in PSA and Free PSA for men with similar types of volume growth. We use standard deviation to define and measure the amount of variation of each type.

Trend and biologic variation may move independently of each other, so we can't simply add them together to produce total variation. The table below shows total standard deviation for PSA Velocity for four trend standard deviations and one biologic standard deviation, assuming they are minimally correlated.

| | | | | |
|---|---|---|---|---|
| Trend Standard Deviation | 0.05 | 0.10 | 0.30 | 0.60 |
| Biologic Standard Deviation | 0.10 | 0.10 | 0.10 | 0.10 |
| Total Standard Deviation | 0.11 | 0.14 | 0.32 | 0.61 |

The results show that the total tends to be dominated by the larger standard deviation, which will usually be the trend standard deviation in the early stages of Dynamic Screening. Even when the component standard deviations are equal at 0.10, the total is only 0.14 rather than the simple sum of 0.20. This result reflects, in part, the independence of the two sources of variation.

Variation in Free PSA Velocity and Free PSA Velocity % trends may be handled in an analogous way to combine trend and biologic standard deviations.

### Residual Values

Maps of residual values and velocities are used to provide early warning of progressing cancer. Residual values are calculated by subtracting predicted values from actual values. Residual velocities can be calculated in several ways. Residual velocities can be calculated by subtracting predicted velocity trends from estimated velocity trends, for example: Residual PSA Velocity (dPSA/dt)=Estimated trend PSA Velocity minus Predicted PSA Velocity. Residual velocities can be calculated by subtracting predicted trends from estimated trends and then calculating the rate of change, for example: Residual PSA Velocity=the annual rate of change in Residual PSA where Residual PSA=Estimated trend PSA minus Predicted PSA trend.

### Residual Value Maps

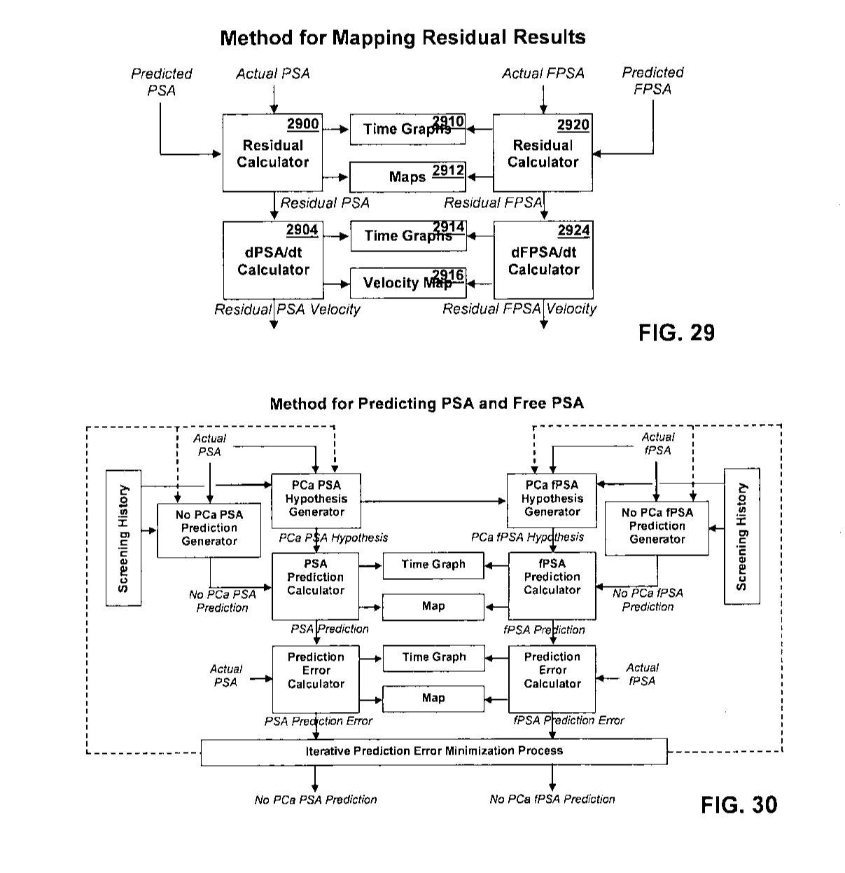

Residual maps of values and velocities may be presented as plots of Free PSA % vs PSA and Free PSA Velocity % vs PSA Velocity, where data points may be determined by the dates of blood tests or spaced by year or some other unit of time. Please refer to FIG. 29 for one way of creating residual value maps.

The residual calculators subtract the predicted value from the estimated trend value of PSA (2900) and Free PSA (2920). Residual PSA, Free PSA and Free PSA % (their ratio) are plotted vs time (2910). Residual Free PSA or Residual Free PSA % is plotted vs residual PSA (2912) for each blood test or for specified dates, perhaps one year apart. Residual PSA Velocity (2904) and Free PSA Velocity (2924) are calculated by differentiating the residual values. Residual PSA Velocity, Residual Free PSA Velocity and Residual Free PSA Velocity % (their ratio) are plotted vs time (2914). Residual Free PSA Velocity or Residual Free PSA Velocity % is plotted vs residual PSA Velocity (2916) for each blood test or for specified dates, perhaps one year apart.

### Alternative Method for Predicting PSA and Free PSA

An alternative method for predicting PSA and Free to be used in calculating residual values is presented in this section. Predicted results are used as a baseline to subtract from actual results to create residual results. The prediction method is introduced below and shown on FIG. 30.

The screening history of all men who have provided data is combined with the screening history of the man making prostate cancer decisions. Possible time paths are generated based on the experience of men with progressing cancer. Time paths for the man without progressing cancer are predicted using a combination of concurrent and sequential methods—which are described in later sections. Predicted values are calculated as the sum of the no cancer prediction and the progressing cancer hypothesis. The error calculators subtract the predicted value from the actual value of PSA and Free PSA. PSA and Free PSA values and prediction errors are plotted vs. time Free PSA is plotted vs. residual PSA for their values and prediction errors. The best prediction is estimated using least squares calculations and other methods to find the prediction that best matches actual results using an iterative survey of a large number of predictions.

### Progressing Cancer

Early detection of progressing cancer is a function of Dynamic Screening.

### Progressing Cancer Trends and Distributions

The Progressing Cancer module (1910) on FIG. 19 considers known trends for progressing cancer. Population studies and other sources are analyzed to predict the time patterns for progressing cancer of PSA Velocity and PSA, Free PSA Velocity and Free PSA, and Free PSA Velocity % and Free PSA %. In addition, the biologic uncertainty in these time patterns is estimated from population studies and other sources.

### Uncertainty in Predicted Values for No Progressing Cancer

The process for estimating the probability of long-term conditions like progressing cancer and volume growth depends on the total amount of uncertainty in the predicted PSA Velocity, Free PSA Velocity and Free PSA Velocity %. The system may estimate uncertainty in the predicted results using standard deviation in the predicted velocities.

Trend uncertainty and biologic uncertainty contribute to the total amount of uncertainty in PSA and Free PSA trends. Trend uncertainty is caused mostly by short-term biologic variation with some test measurement variation thrown in, module 1902 on FIG. 19. The other source of variation is long-term biologic uncertainty about progressing cancer. It reflects variation in PSA and Free PSA for men with similar types of progressing cancer. We use standard deviation to define and measure the amount of variation of each type.

Trend and biologic variation may move independently of each other, so we can't simply add them together to produce total variation. The table below shows total standard deviation for PSA Velocity for four trend standard deviations and one biologic standard deviation, assuming they are minimally correlated.

| | | | | |
|---|---|---|---|---|
| Trend Standard Deviation | 0.05 | 0.10 | 0.30 | 0.60 |
| Biologic Standard Deviation | 0.10 | 0.10 | 0.10 | 0.10 |
| Total Standard Deviation | 0.11 | 0.14 | 0.32 | 0.61 |

The results show that the total tends to be dominated by the larger standard deviation, which will usually be the trend standard deviation in the early stages of Dynamic Screening. Even when the component standard deviations are equal at 0.10, the total is only 0.14 rather than the simple sum of 0.20. This result reflects, in part, the independence of the two sources of variation.

Variation in Free PSA Velocity and Free PSA Velocity % trends may be handled in an analogous way to combine trend and biologic standard deviations.

### Alternative Hypothesis Generators for Progressing Cancer

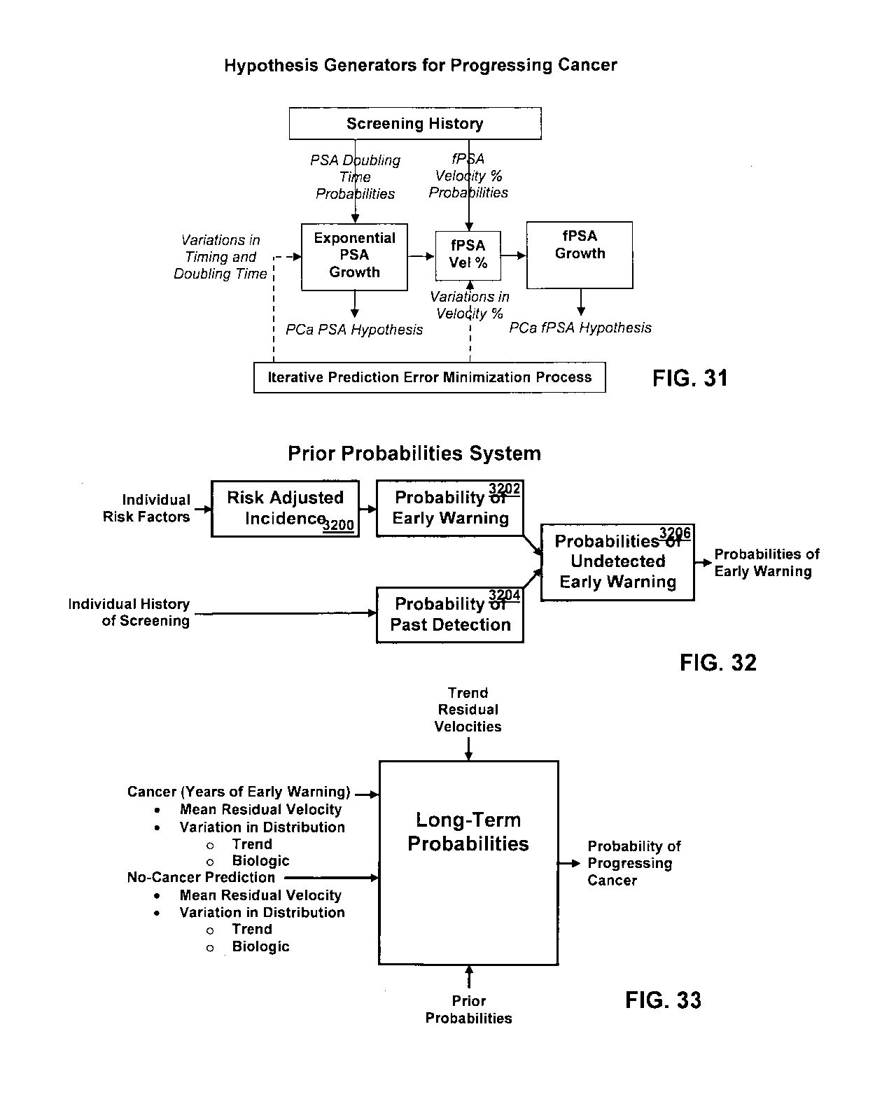

Progressing cancer hypotheses for PSA and fPSA growth may be generated using the screening history of other men with progressing cancer, as shown in FIG. 31. The screening history focuses on the residual values of PSA and fPSA generated by progressing cancer alone without the contributions of non-cancerous prostate cells. PSA doubling times and fPSA Velocity % probabilities are variables used. A doubling time is selected and the exponential growth path for PSA is calculated for each hypothesis generated. A value for fPSA Velocity % is selected for each hypothesis generated. The growth path for fPSA is calculated by combining the fPSA Velocity % with the exponential growth in PSA. PSA timing and doubling times and fPSA Velocity % are varied as part of the iterative error minimization process.

### Early Warning

The Early Warning of progressing cancer module (1918) on FIG. 19 estimates the number of years of early, or late, warning based on the trends for an individual man. Residual PSA Velocity is the preferred trend to use but the PSA Velocity trend or even the PSA trend may be analyzed in conjunction with related Free PSA variables and their ratios with PSA variables. Residual PSA Velocity may be compared with prostate cancer trends that relate PSA Velocity from progressing cancer to the number of years of early warning, allowing Residual PSA Velocity to be translated into an equivalent number of years of early warning. Years of early warning refers to the number of years before the Transition Point when the Cure Ratio begins to decline steeply over time.

### Prior Probabilities

The Prior Probabilities module (1908) on FIG. 19 uses population data, the man's risk factors and his screening history to estimate the probability of undetected early warning. Inputs may include risk factors for the individual being screened and his individual history of screening. More detailed steps of the Prior Probabilities module are shown on FIG. 32.

We define the Transition Point as the year in which cancer has progressed enough to begin causing a steep decline in the Cure Ratio. Early warning is defined as detection before the Transition Point. It is measured in years before the Transition Point. Late warning is defined as detection after the Transition Point.

Risk adjusted means that the risk for an average man has been adjusted up or down by the Risk Ratio entered in the Personal Profile for a specific man. Undetected refers to the probability of cancer that has not already been detected. For cancer with eight years of early warning probability of previous detection is low after many years of Dynamic Screening, so the undetected probability of that early cancer is relatively high. In contrast, for cancer with three years of late warning the probability of previous detection is high, so the undetected probability of that late cancer is very close to zero.

### Risk Adjusted Incidence

The Risk Adjusted Incidence module (3200) on FIG. 32 may estimate the probability of progressing cancer for a range of years of early (or late) warning based on individual risk factors. Men throughout the world may have higher or lower risk than the average man in the United States. Users may input in their personal Profile their personal Risk Factors or their estimate of their Risk Ratio. Background and guidance for choosing a Risk Ratio is provided there. Factors that appear to affect the risk of prostate cancer include: Family history; Race—Black is at risk, possibly because of lack of vitamin D; Diet—Asian is better than American with lots of beef; and Latitude of home that affects sunlight creation of vitamin D.

The Risk Ratio may scale the Average Annual Risk using the following formula:

Risk Adjusted Annual Risk(age)=Risk Ratio×Average Annual Risk(age)

Average Annual Risk is the annual risk for a man of a given age in the reference population, such as all U.S. men. The Risk Ratio may be entered by the user or estimated by the module based on risk factors entered by the user.

### Probability of Early Warning

The Probability of Early Warning module is shown as (3202) on FIG. 32. The module may consider the probability of progressing cancer for each year of early and late warning for him at his current age. Consider a man age 60. At his current age 60, the age 59 Risk Adjusted Incidence of progressing cancer will be one year late (+1). In the same way his age 58 cancer will be two years late (+2) at his current age 60. In the opposite direction, at age 61 his annual risk at the Transition Point will be one year early at his current age 60. In the same way his age 62 cancer will be two years early (−2) at his current age 60. The table below shows the mapping.

| | Years |
|---|---|
| Age | Before/After |
| 58 | +2 |
| 59 | +1 |
| 60 | 0 |
| 61 | −1 |
| 62 | −2 |

One possible equation for the mapping is:

Years Before/After the Transition=Current Age−Age

### Probability of Past Detection

The Probability of Past Detection module is shown as (3204) on FIG. 32. The longer a man uses Dynamic Screening the more early warning of progressing cancer he is likely to get. Past Dynamic Screening increases the chance that more advanced cancer will already have been detected; and, therefore, is no longer a likely possibility.

The probability of detection increases with later warning. In the extreme, metastasis and death unambiguously confirm the detection of prostate cancer. Symptoms typically show up at about three or four years of late warning, so much of this cancer will be detected in men who are not screened. Current PSA screening is hit or miss, but tends to detect cancer in a range around the Transition Point (year 0). These situations are taken into account by the module when it estimates the probability of past detection as a function of early warning.

Two inputs are used for the most basic estimates for past Dynamic Screening: Years of PSA Dynamic Screening and Years of Free PSA Dynamic Screening. Longer periods of testing lead to earlier warning of progressing cancer. There can be a matrix of possible past detection vectors based on these two dimensions. The probability of past detection varies greatly depending on the type and duration of screening.

Some men may continue Dynamic Screening after the apparent detection of progressing cancer in order to be sure that cancer is progressing. This situation requires special handling to reflect possible detection that has not been acted on.

### Probabilities of Undetected Early Warning

The Probability of Undetected Early Warning module is shown as (3206) on FIG. 32. The probability of undetected early warning is a function of the probability of early warning (3202) and the probability of past detection (3204). One possible equation is:

Probability of Undetected Early Warning(years before/after Transition Point)=Probability of Early Warning(years)×(1−Probability of Past Detection (years))

### Long-Term Probabilities

The Long-Term Probabilities module (1916) on FIG. 19 estimates the probabilities of one or more long-term conditions, such as progressing cancer or prostate volume growth. FIG. 33 shows an example of the high level inputs and outputs for estimating the probability of progressing cancer. Prior probabilities are the starting point and come from module 1908 on FIG. 19. Trend residual velocities come from module 1912 on FIG. 19. Velocities and trends may be used in other embodiments. The Long-Term Probabilities module on FIG. 33 adjusts the prior probabilities of progressing cancer based on how the trend residual velocities compare with patterns for progressing cancer and the predicted values for no cancer. A variety of methods can be used to estimate the probability, including Bayesian and simulation methods. The process is complicated because a variety of cancer stages are possible, characterized by years of early warning. Therefore, the module may consider a range of progressing cancer possibilities (different years of early warning) and a no-cancer (not present or not progressing) possibility defined by the no-cancer predicted values. For each of these possibilities a probability distribution may be constructed that may be characterized by a mean and by variation, which may be characterized by standard deviations. There are two sources of variation that may be considered. First, trend variation may be caused by possibly random variation in test results. Second, biologic variation may be caused by differences among men or for a specific man over time.

### Example Flow Chart for Direct Calculation of Probabilities

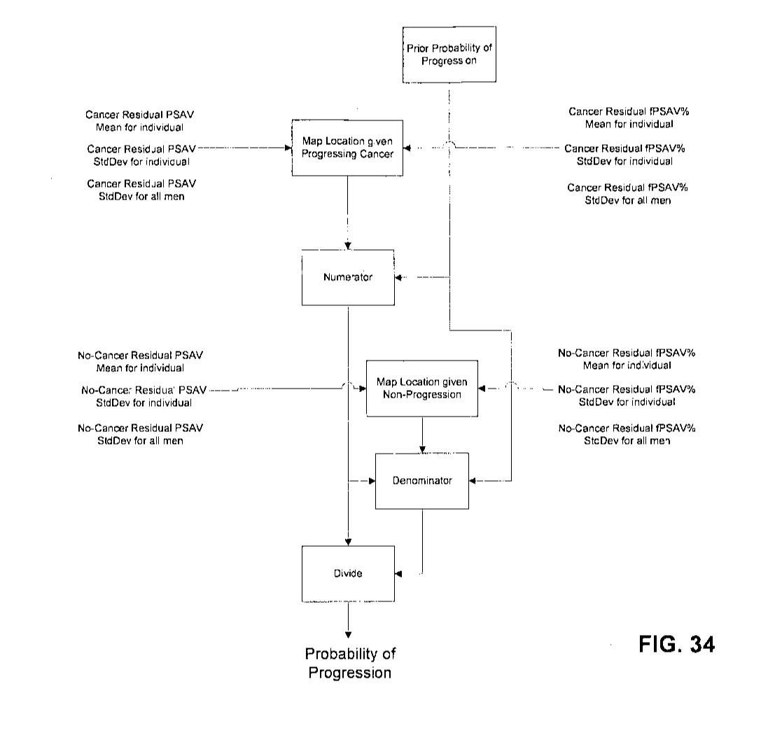

A high level flow chart of the direct calculation of the probability of progressing cancer is shown on FIG. 34. The direct calculation may be based on Bayesian methods and contrasts with iterative methods.

### Probability Estimate for Iterative Calculation of Probabilities

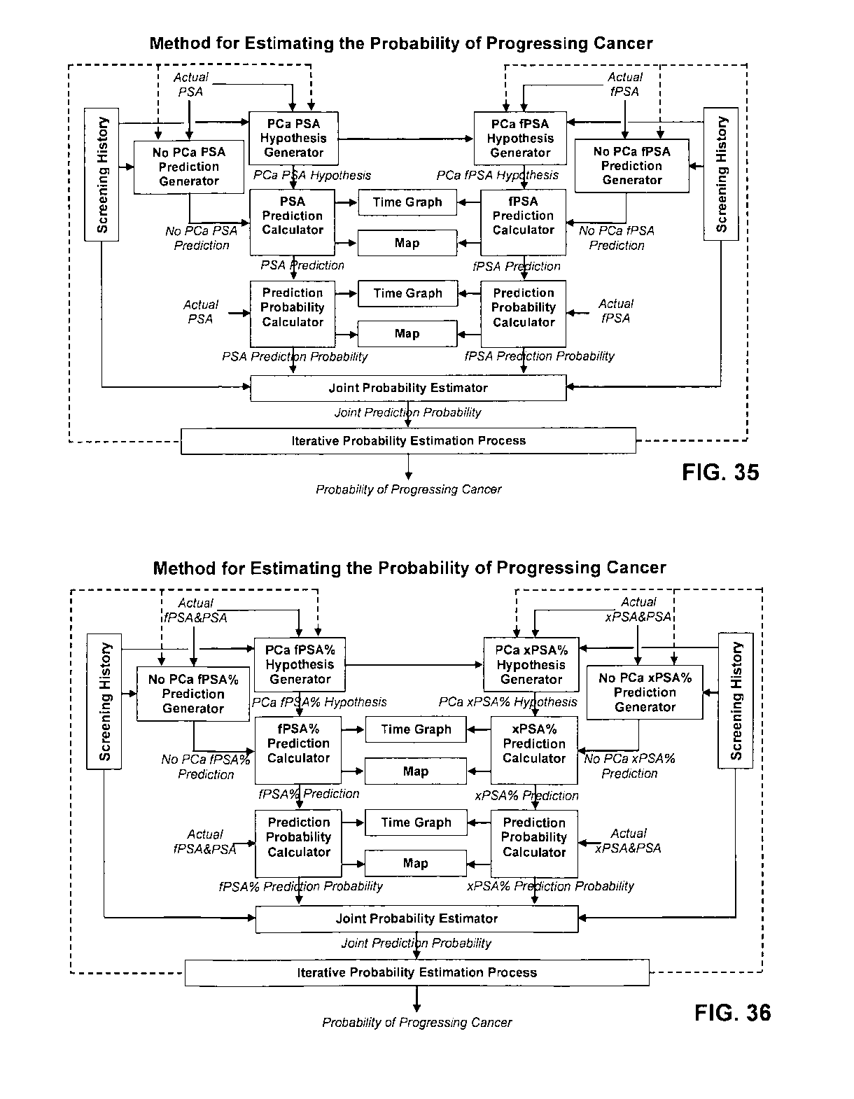

An iterative process may be used to calculate the probability of progressing cancer. Estimates of the probability distributions of the components that comprise actual and predicted PSA and Free PSA are used to generate probability distributions used in the method. The method is shown by the flow chart on FIG. 35. The process iteratively selects predictions and hypotheses, calculates their probabilities and then through a series of steps calculates the joint probability of the resulting prediction. At the end of the iterative process the probability of progressing cancer is calculated.

The screening history of all men who have provided data is combined with the screening history of the man making prostate cancer decisions. Variables considered include: No Cancer and Cancer PSA and fPSA Trends, including: Average PSA and fPSA % trends, PSA Velocity and fPSA Velocity % trends, and Residual PSA and fPSA Velocity % trends. Probabilities are associated with various combinations of PSA doubling time and fPSA Velocity % from cancer based on the experience of men with progressing cancer. The probability distributions of the components of predicted PSA and Free PSA are estimated—volume measurement error and velocity density prediction errors are example factors that can cause PSA to vary. The error distributions are run through a prediction simulator to translate the input error distributions into an overall probability distribution for predicted PSA and Free PSA for no progressing cancer. Predictions of PSA and fPSA are calculated by adding the progressing cancer hypothesis to the no cancer prediction. The joint probability for each prediction is calculated from the probabilities for each progressing cancer hypothesis and no cancer prediction. PSA and Free PSA values and prediction errors are plotted vs time. Free PSA is plotted vs residual PSA for their values and prediction errors. The joint probability that the actual results are explained by the predictions is calculated using a variety of methods, including Bayesian inference. The probability of progressing cancer is estimated after many iterations of hypotheses and predictions based on the probabilities of scenarios with progressing cancer.

### Probability Estimate Using Confirming Tests

The system will recognize early warning of possible cancer progression and suggest additional confirmation tests. Confirmation tests may include other components of PSA such as Pro PSA and any other useful new markers developed in the future. In addition, a new prostate volume study may be suggested, perhaps using more expensive technology if rapid prostate enlargement is a factor. A second round of confirmation tests will be suggested—perhaps six months after the first. Additional confirmation tests will be suggested until progression has been confirmed or rejected. The initial drop in Free PSA % ratios may be an accident, but a continued drop accompanied by shifts in ratios for other tests for can confirm a high probability of progressing cancer. Recall the adage that once may be an accident, twice a coincidence but three times is enemy action—with prostate cancer as the enemy in this case.

A possible confirming method is shown on the flow chart on FIG. 36. The process iteratively selects predictions and hypotheses, calculates their probabilities and then through a series of steps calculates to joint probability of the resulting prediction. At the end the iterative process the probability of progressing cancer is calculated from the scenarios that include it.

The screening history of all men who have provided data is combined with the screening history of the man making prostate cancer decisions. Variables considered include: No Cancer and Cancer fPSA, xPSA and PSA Trends, including: Average fPSA %/xPSA % trends, fPSA/xPSA Velocity % trends, and Residual fPSA/xPSA Velocity % trends. Probabilities are associated with various combinations of PSA doubling time and fPSA and xPSA Velocity % s from cancer based on the experience of men with progressing cancer. Bayesian inference may be used to estimate the probability distributions of the components of predicted PSA, fPSA and xPSA—volume measurement error and velocity density prediction errors are example factors that can cause PSA to vary. The error distributions are run through a prediction simulator to translate the input error distributions into an overall probability distribution for predicted PSA, fPSA and xPSA for no progressing cancer. Predictions of PSA, fPSA and xPSA are calculated by adding the progressing cancer hypothesis to the no cancer prediction. The joint probability for each prediction is calculated from the probabilities for each progressing cancer hypothesis and no cancer prediction. PSA, fPSA and xPSA values and prediction errors are plotted vs time. Residual fPSA and xPSA are plotted vs residual PSA for their values and prediction errors. The joint probability that the actual results are explained by the predictions may be calculated using Bayesian methods. The probability of progressing cancer is estimated after many iterations of hypotheses and predictions based on the probabilities of scenarios with progressing cancer.

### Warnings and Alerts

Cancer warnings and alerts may be triggered by variables in the Dynamic Screening Analysis System and may determine choices of custom content. Cancer warnings may be triggered when a combination of the probability of progressing cancer and the years of early warning reach predetermined levels. Cancer alerts may be triggered when a combination of residual velocities and strength of evidence reach predetermined levels.

### Green, Caution and Stop Cases

One or two anomalous tests can skew trends and temporarily push results into an Alert status or even a Warning status—especially if the user is just starting Dynamic Screening or is testing infrequently. The Dynamic Screening System helps assess the impact of potentially anomalous tests by presenting results for additional cases where one or two of the most anomalous results are excluded—the Yellow Caution and Green cases. The Green Case excludes the two Test Pairs that most increase concern about progressing cancer. It provides the least early warning with the least overstatement of risk. The Yellow Caution Case urges caution before drawing any conclusions from this case. It excludes the test pair that causes most concern about progressing cancer. It provides earlier warning with more potential overstatement of risk than the Green case. The Red Stop Case urges users to stop and pause before drawing any conclusions from this case. It provides the earliest warning but may overstate the risk based on only one or two tests. The Red Stop case doesn't exclude any tests, other than tests excluded because they are outside the tolerance area False Alerts from minor infections of the prostate are most likely for this case.

### Cancer Warnings

Cancer warning status may determine custom content in reports to users. Warning levels may be triggered when specified variables reach predetermined levels, either individually or in combination. Variables that may trigger cancer warnings include the probability of progressing cancer and the number of years of early warning.

### Cancer Alerts

Cancer Alert status signals some concern about test trends and raises the question: How much sooner than one year should the next Test Pair (PSA+Free PSA) be scheduled? The Alert is likely to be caused by random variation or a mild infection of your prostate. In rare cases, it may be a very early hint of progressing cancer. Alert levels may be triggered when specified variables reach predetermined levels, either individually or in combination. Variables that may trigger Alerts include residual values such as residual PSA Velocity and residual Free PSA Velocity % and the strength of evidence based on variables such as the length of screening history and the number of screening tests of each type, for example PSA and Free PSA.

The Alert status has been triggered by an increase in the Alert level for at least one case on the graph at the bottom left. Alert levels are based on dynamic analysis of PSA and Free PSA trends, as shown later. Alert levels increase on a scale of 1 to 10 as trends look more like progressing cancer and less like volume growth. The seriousness of the Alert increases as the strength of test evidence increases, shown on the graph at the bottom right. Strength of Evidence increases with more tests over a longer period of time, as explained later. The Residual Velocity Map captures this information to create a picture of prostate cancer if it is progressing. Velocities are the annual change in each variable. Residual velocities are the annual changes from progressing cancer (in theory) after estimates of velocities caused by benign volume growth are subtracted. Residual PSA Velocity is the horizontal axis. Residual Free PSA Velocity % is the vertical axis—calculated as residual Free PSA Velocity divided by residual PSA Velocity. It is an attempt to measure the Free PSA % from new progressing cancer or unexpected prostate volume growth. Predetermined curves on the residual velocity map may be used to determine Alert levels either on their own or in combination with other variables, such as strength of evidence.

### Custom Content System

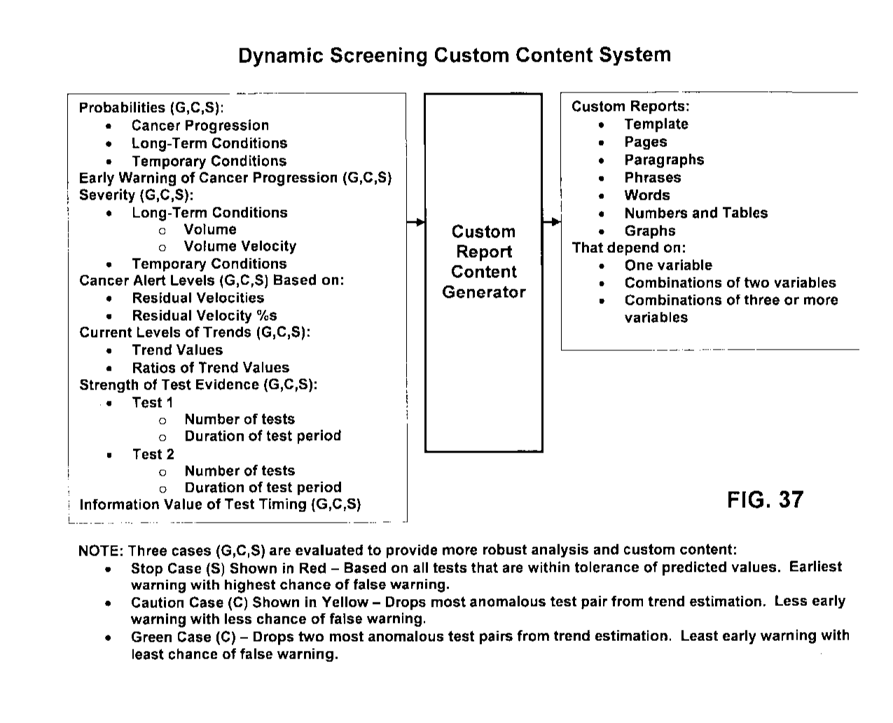

A high level block diagram of how the custom content system might function is shown on FIG. 37. Custom content includes words, paragraphs, numbers, tables, graphs and other content used in custom reports produced by the system and suggested by the list of outputs on the right of FIG. 37. Custom content can depend on one input variable or combinations of two or more variables suggested by the list of input variables on the left. Custom content may take into account variations among the variables for the three cases: Red Stop, Yellow Caution and Green.

An example of custom content based on two variables is shown below with brief custom content shown in italics below each combination of probability of progressing cancer and length of early warning of progressing cancer:

If Low probability of progressing cancer and Long early warning then content is:
Wait patiently as continued testing decreases or increases the probability.
   If High probability of progressing cancer and Long early warning then content is:
Explore treatments and timing in a deliberate manner because you have time.
   If Low probability of progressing cancer and Short early warning then content is:
Test intensively because time is short in the unlikely event cancer is progressing.
   If High probability of progressing cancer and Short early warning then content is:
Schedule best treatment quickly because you are short of time.

### VI. Feedback Learning

Feedback can be a part of improving the accuracy and reliability of one or more of the disclosed systems and methods. Evaluation of the experience of many men using disclosed approaches will provide better estimates of the values and probabilities of many of the variables used in the analysis. The results of each individual evaluation are combined with others and analyzed as a group to create summaries of all screening histories.

It can be less difficult to evaluate individual experience looking backward than it is to predict it looking forward. For example, looking backward allows us to separate individuals into two groups: men who have experienced progressing cancer and men who have not. This knowledge removes an uncertainty from the analysis and allows precise estimation of the contributions of progressing cancer and other factors like enlargement due to BPH.

Improving our ability to predict outcomes and estimate the probability distributions of those outcomes is a central part of the feedback learning process. Multi-dimensional response surfaces will be developed where possible to fine tune the predictions and estimates based on a variety of variables that may include age, race and other demographic variables. Response surfaces will be estimated using standard statistical methods, such as multiple regression analysis. They will be used for two groups of men: Men without Progressing Cancer who may be affected by Infections, Related Changes, and Enlargement from BPH; and Men with Progressing Cancer.

Here are two examples of what we expect to learn. For men without progressing cancer, the stability of the velocity densities is a determinant of our confidence in the predictions of PSA and Free PSA. We expect to learn more about how it behaves through feedback learning. For men with progressing cancer, the joint probability of concurrent changes in the residual Free PSA Velocity % and similar variables improves our confidence in early warning. We expect to learn more about how they are correlated through feedback learning.

### Overall and Detailed Feedback Learning

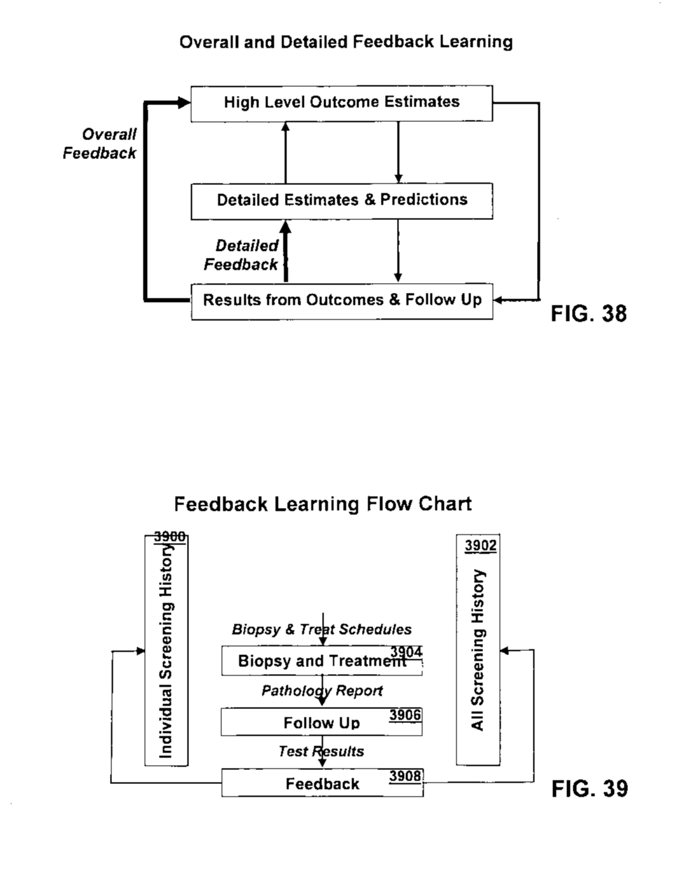

Two types of feedback learning will improve the method over time, as suggested by the flow chart on FIG. 38. Detailed feedback will improve the accuracy of estimates and predictions. Overall feedback will allow us to make sure that estimates of high level outcomes based on detailed estimates and predictions will be unbiased and consistent with overall results. Two examples of high level outcomes are progression probability and Cure Ratio. We will focus on them in much of the following discussion.

Detailed Feedback will be collected for every variable (or important variable) used in the estimation and prediction process. Best estimates and probability distributions will be calculated and used in the estimation and prediction parts of the method. For example, PSA and Free PSA velocity density may be considered important variables used in the prediction process for progression probability, as noted earlier. The probability distributions for predictions depend on how much those variables are likely to vary from year to year for a given man. Less variation for a wide range of men means a tighter probability distribution around the predictions based on those variables.

Overall Feedback calibrates the method so that estimates of high level outcomes using detailed methods are consistent with actual high level outcomes for groups of the population. For example, the average estimated probability for the whole population based on detailed methods should be consistent with the overall probability for the whole population. In addition, this consistency should be maintained for smaller groups of the population.

### Information Gathering

The feedback process depends on gathering information about outcomes, as suggested by FIG. 39. Information about outcomes can be fed back to Individual Screening History (3900) and to All Screening History (3902) for analysis of groups of individuals.

The Biopsy and Treatment module is 3904 on FIG. 39. For a biopsy, a doctor uses a device to inject thin hollow needles into the prostate to extract tissue. A pathologist exams the tissue and provides a diagnosis of prostate cancer if it exists. Primary treatment is intended to cure prostate cancer. It includes surgery to remove the prostate and various types of radiation to kill the cancer. A pathology report after surgery can provide useful information about the progress of cancer. The results of these pathology reports will provide useful feedback about outcomes that will allow us to improve the effectiveness of the method.

The Follow Up module is 3906 on FIG. 39. PSA tests and periodic physicals are used to follow patients' progress after treatment or no treatment depending on their choice. PSA tests are used to determine recurrence and the early progress of the disease. Later symptoms, metastasis and eventually death will be followed for many men. Feedback of these outcomes will help us improve the effectiveness of the method, as outlined in the next section.

The Feedback module is 3908 on FIG. 39. Decisions and results for each man can be analyzed to learn what actually happened. The results can be pooled with others and analyzed for common trends and probability distributions of outcomes. The distributions can be combined with information from a single man to improve predictions and estimates of probabilities, especially for progression.

### High Level Outcomes

There are a range of high level outcomes, including progression probability, Cure Ratio, metastasis and death from prostate cancer and side effects of treatment. We will focus on examples for progression probability, predictions and Cure Ratio.

### Feedback for Progression Probability

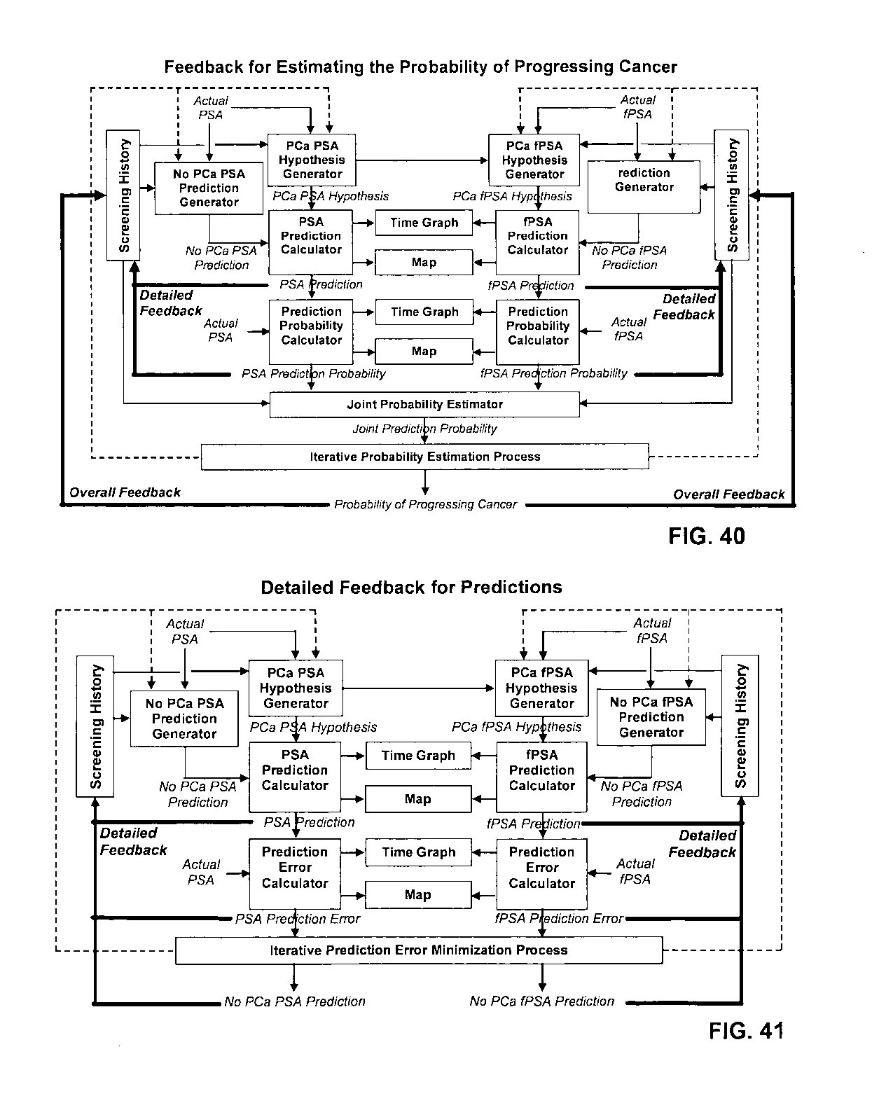

We will discuss how we use feedback from two types of primary tests to estimate progression probability: PSA and Free PSA tests and additional confirming tests. The flow chart on FIG. 40 suggests: How the probability of progressing cancer is calibrated using overall feedback from many men, and How detailed feedback is used to improve predictions and estimates of the probability distributions of the predictions.

### Detailed Feedback for Predictions

Predictions of PSA and Free PSA, and hypotheses about their production by cancer, can be used for estimating the probability of progression, as we have seer. Detailed feedback about predictions of PSA and Free PSA and the associated prediction error are the starting point for improving predictions, as suggested by the flow chart on FIG. 41.

### Detailed Feedback for No Progressing Cancer

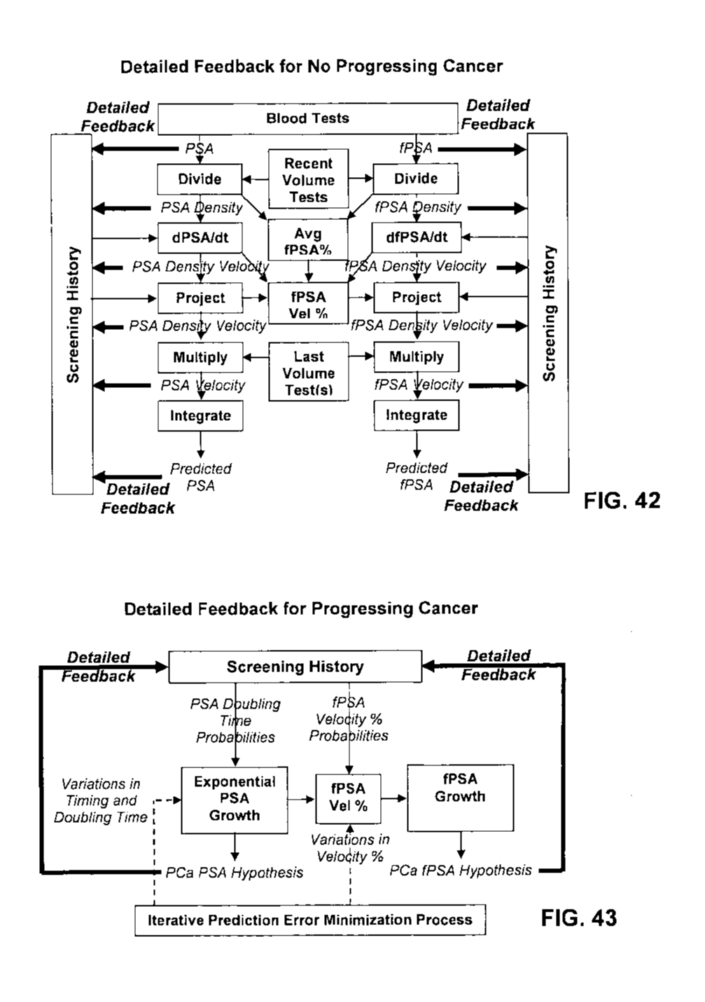

Detailed feedback is analyzed for every variable of the prediction process for no progressing cancer, as suggested on FIG. 42. We have already mentioned the importance of density velocity and how its stability is likely to allow accurate predictions. Detailed feedback from other variables is also likely to turn out to be helpful in improving the method.

### Detailed Feedback for Infections

Detailed feedback will help improve our ability to identify test results distorted by infections of the prostate. Feedback about PSA, Free PSA and other test results will be analyzed for men who have been diagnosed with infections, possibly using the feedback shown on FIG. 42.

### Detailed Feedback for Related Changes

Detailed feedback will help improve our ability to analyze how test results are distorted by related changes in diet, treatment, medication and other factors. Feedback about PSA, Free PSA and other test results will be analyzed for men who have made changes in one or more of these factors with the goal of improving our ability to predict the impact of the changes in other men, possibly using the feedback shown on FIG. 42.

### Detailed Feedback for Progressing Cancer

Detailed feedback is analyzed for every variable of the hypothesis generation process for progressing cancer, as suggested on FIG. 43. Variables include PSA doubling time and variations in the Free PSA velocity %. Analysis of the probability distributions of these and other variables will be used in the higher level process of estimating the probability of progressing cancer.

Feedback from Confirming Tests

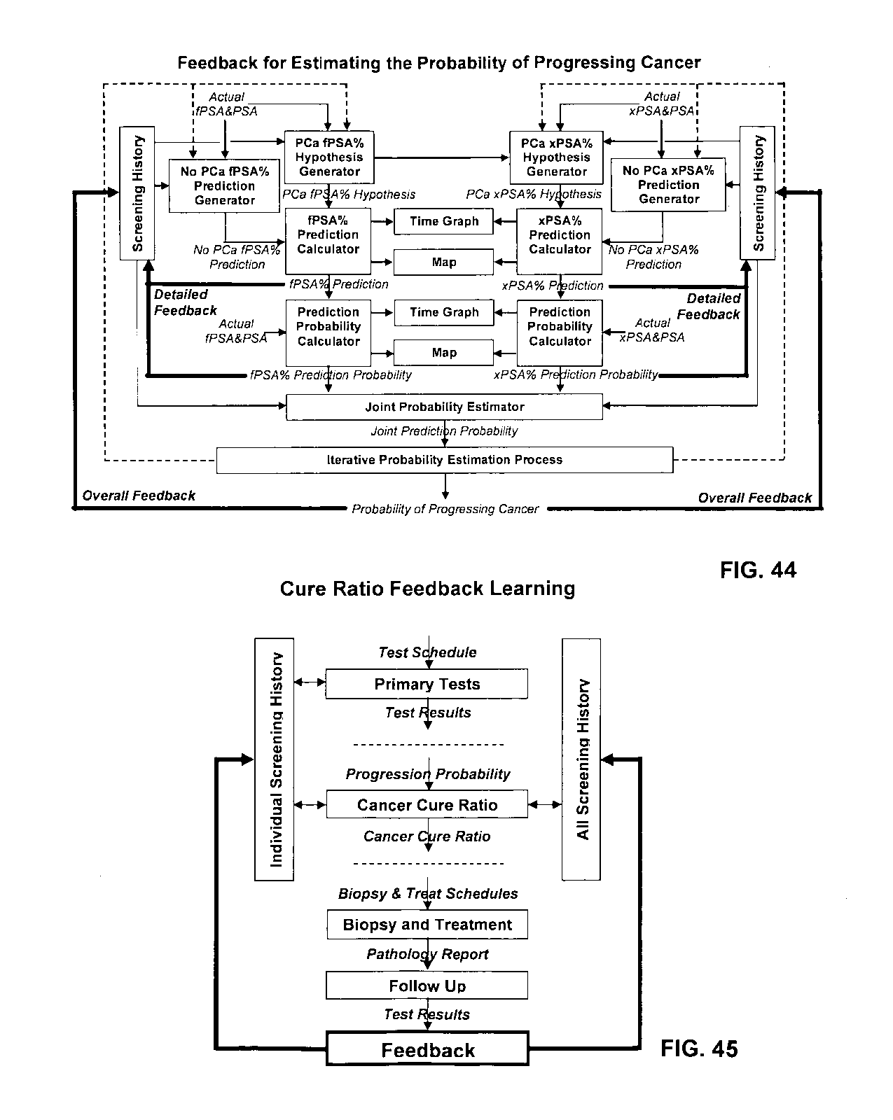

Previous sections have outlined how PSA and Free PSA are predicted, analyzed and combined to predict ratios. Those sections are summarized on the left side of the flow chart on FIG. 44. A similar process is carried out for PSA and one or more additional variables, as summarized on the right side of the flow chart using the general term xPSA. The broad goal of this feedback process is to find one or more confirming tests that when combined with PSA and Free PSA will provide strong confirmation of early warning of progressing cancer. The flow chart on FIG. 44 suggests: How feedback from a range of confirming tests is used to estimate the probability of progressing cancer using overall feedback from many men; and How detailed feedback is used to improve predictions and estimates of the probability distributions of the predictions.

Estimates of Cure Ratio used in the method will also be improved by feedback. The high level flow chart on FIG. 45 suggests how feedback about a variety of outcomes will be used to improve estimates of the Cure Ratio as a function of primary results. Primary test results will be related to the pathology results after biopsy and treatment (if surgery) and eventually to the probability of recurrence, metastasis and eventually death. For example, the results of late detection are likely to lead less favorable pathology (perhaps Stage T2 and Gleason 7 or more), more frequent recurrence, metastasis and eventually death. In contrast, the results of early detection are likely to lead to favorable pathology (Stage T1, Gleason 6 or more, and small cancer volumes).

### Feedback for Estimating Cure Ratio

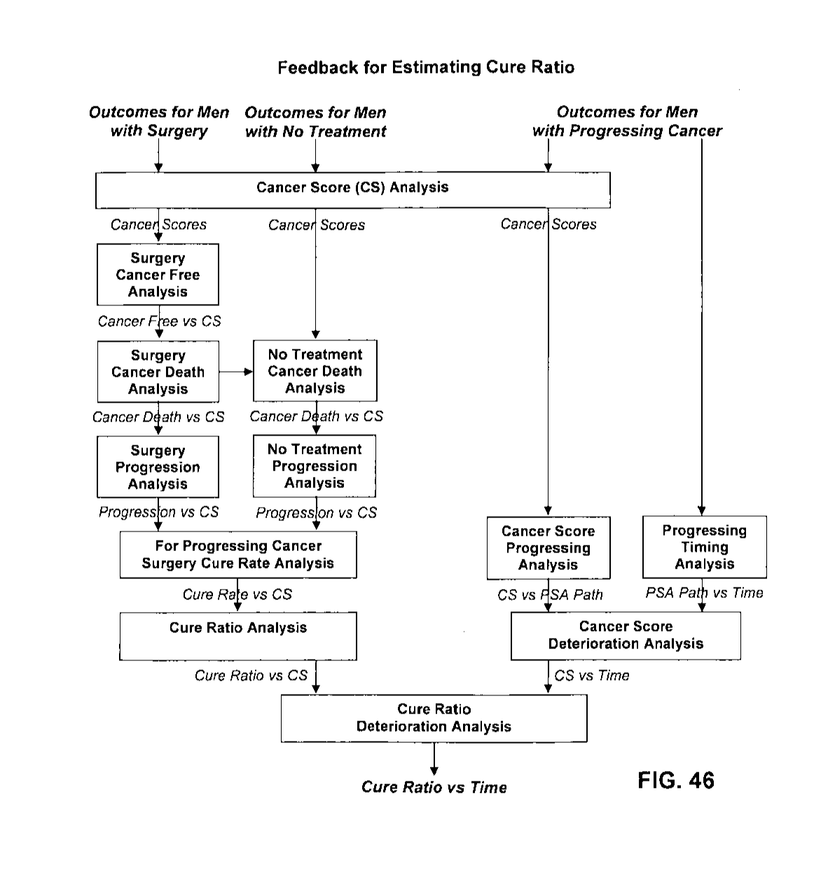

Details of how feedback will be used to estimate Cure Ratio are shown on the flow chart on FIG. 46. Outcomes for men with surgery, no treatment and progressing cancer will be used to supplement and eventually replace the results from studies reported in medical journals. The Cancer Score will be estimated for men in each group and feedback about the corresponding results will be used to improve each step in the calculation of Cure Ratio.

With respect to this disclosure, while examples have been used to disclose the invention, including the best mode, and also to enable any person skilled in the art to make and use the invention, the patentable scope of the invention is defined by claims, and may include other examples that occur to those skilled in the art. Accordingly the examples disclosed herein are to be considered non-limiting. As an illustration, it should be understood that for the processing flows described herein, the steps and the order of the steps may be altered, modified, removed and/or augmented and still achieve the desired outcome. A multiprocessing or multitasking environment could allow two or more steps to be executed concurrently.

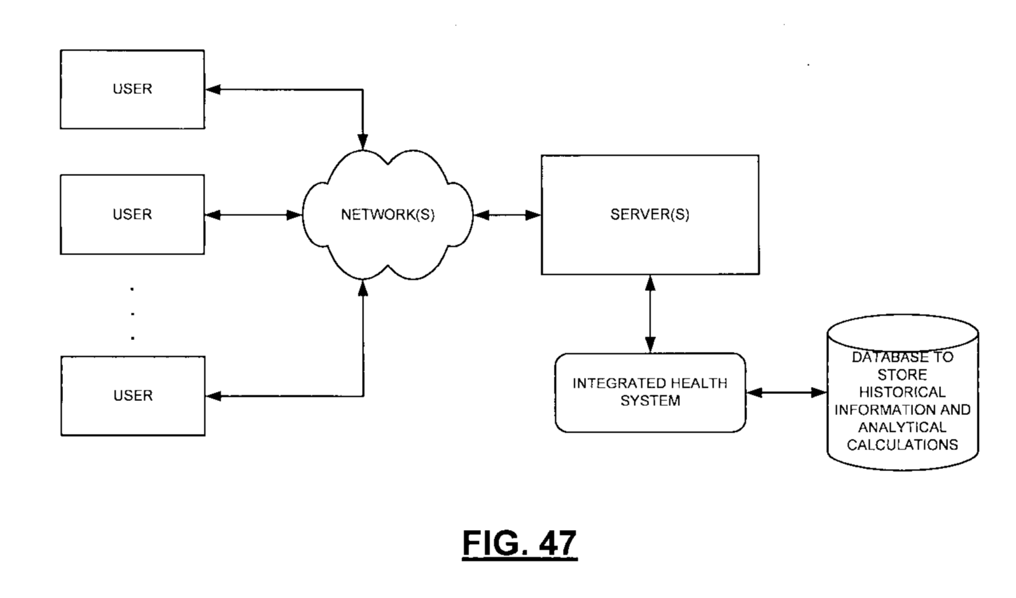

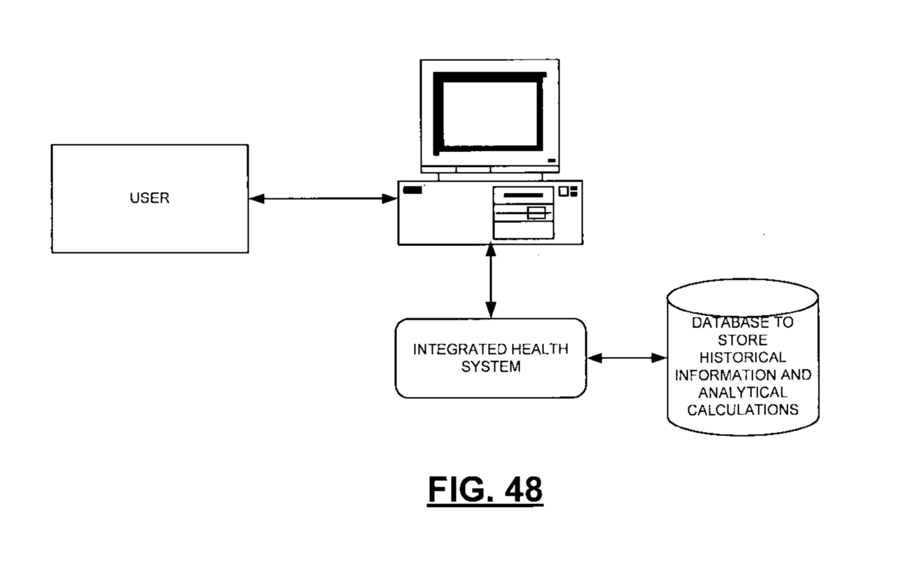

It is further noted that the systems and methods may be implemented on various types of computer architectures, such as for example on a networked system (e.g., FIG. 47), or in a client-server configuration, or in an application service provider configuration, on a single general purpose computer or workstation (e.g., FIG. 48), etc. The systems and methods may include data signals conveyed via networks (e.g., local area network, wide area network, Internet, combinations thereof, etc.), fiber optic medium, carrier waves, wireless networks, etc. for communication with one or more data processing devices. The data signals can carry any or all of the data disclosed herein (e.g., user input data, the results of the analysis to a user, etc.) that is provided to or from a device.

Additionally, the methods and systems described herein may be implemented on many different types of processing devices by program code comprising program instructions that are executable by the device processing subsystem. The software program instructions may include source code, object code, machine code, or any other stored data that is operable to cause a processing system to perform methods described herein.

The systems' and methods' data (e.g., associations, mappings, etc.) may be stored and implemented in one or more different types of computer-implemented ways, such as different types of storage devices and programming constructs (e.g., data stores, RAM, ROM, Flash memory, flat files, databases, programming data structures, programming variables, IF-THEN (or similar type) statement constructs, etc.). It is noted that data structures describe formats for use in organizing and storing data in databases, programs, memory, or other computer-readable media for use by a computer program.

The systems and methods may be provided on many different types of computer-readable media including computer storage mechanisms (e.g., CD-ROM, diskette, RAM; flash memory, computer's hard drive, etc.) that contain instructions (e.g., software) for use in execution by a processor to perform the methods' operations and implement the systems described herein.

The computer components, software modules, functions, data stores and data structures described herein may be connected directly or indirectly to each other in order to allow the flow of data needed for their operations. It is also noted that a module or processor includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality may be located on a single computer or distributed across multiple computers depending upon the situation at hand.

1.-14. (canceled)

15. A method of treating prostate cancer in a subject, the method comprising:
(a) measuring levels of a plurality of biomarkers in samples obtained from the subject at different times;
(b) measuring prostate volume of the subject at one or more times;
(c) calculating initial fitted trends for the plurality of biomarkers and the prostate volume;
(d) for each fitted trend of biomarker levels, removing a biomarker level outside a tolerance region;
(e) calculating a new fitted trend for the plurality of biomarkers and the prostate volume based on biomarker levels remaining after step (d);
(f) repeating steps (d) and (e) to produce final fitted trends, wherein remaining biomarker levels are within the tolerance region;
(g) calculating a probability of progressing prostate cancer in the subject based on a comparison between the final fitted trends and population data for levels of the biomarkers and prostate volume; and
(h) treating the subject based on results of step (g);
wherein steps (c) to (g) are performed using a computer system.

16. The method of claim 15, wherein the plurality of biomarkers are selected from the group consisting of: PSA, free-PSA (fPSA), and a ratio one of these biomarkers to the other.

17. The method of claim 15, wherein the plurality of biomarkers comprise PSA and fPSA.

18. The method of claim 15, wherein the plurality of biomarkers comprise PSA and fPSA %, wherein fPSA % is fPSA divided by PSA.

19. The method of claim 15, wherein the trend of prostate volume comprises volumes estimated based on a biomarker trend.

20. The method of claim 15, wherein prostate volume is measured at a plurality of times.

21. The method of claim 15, wherein calculating the probability comprises calculating a trend velocity for a final fitted trend.

22. The method of claim 21, wherein the trend velocity is PSA velocity.

23. The method of claim 15, wherein treating the subject comprises performing a biopsy.

24. The method of claim 15, wherein treating the subject comprises surgery or radiation exposure.

25. The method of claim 15, further comprising diagnosing prostate cancer in the subject based on the probability calculated in step (g).

26. The method of claim 15, wherein the biomarker removed from a trend in step (d) is the biomarker farthest from the tolerance region.

27. The method of claim 15, wherein timing of the treatment is selected based on the probability of progressing cancer.
